(19) United States
(12) Patent Application Publication
(10) Pub. No.: US 2013/0172688 A1
(43) Pub. Date: Jul. 4, 2013

(54) DIABETES MANAGEMENT APPLICATION FOR MOBILE PHONE

(71) Applicants: Janette E. Allen, Zionsville, IN (US); Eric S. Carlsgaard, Zionsville, IN (US); Paul J. Galley, Cumberland, IN (US); Robert E. Reinke, Indianapolis, IN (US); Joe B. Thomas, Greenfield, IN (US)

(72) Inventors: Janette E. Allen, Zionsville, IN (US); Eric S. Carlsgaard, Zionsville, IN (US); Paul J. Galley, Cumberland, IN (US); Robert E. Reinke, Indianapolis, IN (US); Joe B. Thomas, Greenfield, IN (US)

(73) Assignee: ROCHE DIAGNOSTICS OPERATIONS, INC., Indianapolis, IN (US)

(21) Appl. No.: 13/461,974

(22) Filed: May 2, 2012

Related U.S. Application Data
(60) Provisional application No. 61/581,159, filed on Dec. 29, 2011.

(51) Int. Cl. A61B 5/0205; A61B 5/145

(52) U.S. Cl. 600/301; 600/365

(57) ABSTRACT

A mobile device for managing diabetes care of a patent is disclosed. The device has a data entry interface configured to receive glucose measures for the patient and store the glucose measures in a patient log residing on the device. The device has a selection module that operates to selectively analyze the glucose measure in the patient log and select a given structural collection procedure from a plurality of standardized collection procedures.

### CROSS-REFERENCE TO RELATED APPLICATIONS

This application claims the benefit of U.S. Provisional Application No. 61/581,159, filed on Dec. 29, 2011. The entire disclosure of the above application is incorporated herein by reference.

### FIELD

The present disclosure relates to a mobile computing device for managing diabetes care of a patient and, more particularly, to a mobile computing device which administers structured collection procedures for patient care indicators and transmits them via a wireless network to a medical care provider.

### BACKGROUND

Diabetes mellitus, often referred to as diabetes, is a chronic condition in which a person has elevated blood glucose levels that result from defects in the body's ability to produce and/or use insulin. There are three main types of diabetes. Type 1 diabetes usually strikes children and young adults, and may be autoimmune, genetic, and/or environmental. Type 2 diabetes accounts for 90-95% of diabetes cases and is linked to obesity and physical inactivity. Gestational diabetes is a form of glucose intolerance diagnosed during pregnancy and usually resolves spontaneously after delivery.

In 2009, according to the World Health Organization, at least 220 million people worldwide suffer from diabetes. In 2005, an estimated 1.1 million people died from diabetes. Its incidence is increasing rapidly, and it is estimated that between 2005 and 2030, the number of deaths from diabetes will double. In the United States, nearly 24 million Americans have diabetes with an estimated 25 percent of seniors age 60 and older being affected. The Centers for Disease Control and Prevention forecast that one in three Americans born after 2000 will develop diabetes during their lifetime. The National Diabetes Information Clearinghouse estimates that diabetes costs $132 billion in the United States alone every year. Without treatment, diabetes can lead to severe complications such as heart disease, stroke, blindness, kidney failure, amputations, and death related to pneumonia and flu.

Management of diabetes is complex as the level of blood glucose entering the bloodstream is dynamic. Variation of insulin in the bloodstream that controls the transport of glucose out of the bloodstream also complicates diabetes management. Blood glucose levels are sensitive to diet and exercise, but also can be affected by sleep, stress, smoking, travel, illness, menses, and other psychological and lifestyle factors unique to individual patients. The dynamic nature of blood glucose and insulin, and all other factors affecting blood glucose, often require a person with diabetes to forecast blood glucose levels. Therefore, therapy in the form of insulin or oral medications, or both, can be timed to maintain blood glucose levels in an appropriate range.

Management of diabetes is often highly intrusive because of the need to consistently obtain reliable diagnostic information, follow prescribed therapy, and manage lifestyle on a daily basis. Daily diagnostic information, such blood glucose, is typically obtained from a capillary blood sample with a lancing device and is then measured with a handheld blood glucose meter. Interstitial glucose levels may be obtained from a continuous glucose sensor worn on the body. Prescribed therapies may include insulin, oral medications, or both. Insulin can be delivered with a syringe, an ambulatory infusion pump, or a combination of both. With insulin therapy, determining the amount of insulin to be injected can require forecasting meal composition of fat, carbohydrates and proteins along with effects of exercise or other physiologic states. The management of lifestyle factors such as body weight, diet, and exercise can significantly influence the type and effectiveness of a therapy.

Management of diabetes involves large amounts of diagnostic data and prescriptive data that are acquired from medical devices, personal healthcare devices, patient recorded information, healthcare professional tests results, prescribed medications and recorded information. Medical devices including self-monitoring blood glucose (bG) meters, continuous glucose monitors, ambulatory insulin infusion pumps, diabetes analysis software, and diabetes device configuration software each of which generates or manages or both large amounts of diagnostic and prescriptive data. Personal healthcare devices include weight scales, and blood pressure cuffs. Patient recorded information includes information relating to meals, exercise and lifestyle. Healthcare professional biomarker data includes glycated hemoglobin (HbA1C), cholesterol, triglycerides, and glucose tolerance. Healthcare professional recorded information includes therapy and other information relating to the patient's treatment.

There is a need for a handheld patient device to aggregate, manipulate, manage, present, and communicate diagnostic data and prescriptive data from medical devices, personal healthcare devices, patient recorded information, biomarker information and recorded information in an efficient manner to improve the care and health of a person with diabetes, so the person with diabetes can lead a full life and reduce the risk of complications from diabetes.

There is a need for a patient to be able to manage, manipulate and control the desired range of blood glucose levels over a period of time through a hand-held device in an efficient manner to improve the care and health of a person with diabetes, so the person with diabetes can lead a full life and reduce the risk of complications from diabetes.

Management of diabetes involves large amounts of diagnostic data and prescriptive data that are acquired from medical devices, personal healthcare devices, patient recorded information, healthcare professional tests results, prescribed medications and recorded information. Clinicians generally treat diabetic patients according to published therapeutic guidelines such as, for example, Joslin Diabetes Center & Joslin Clinic, Clinical Guideline for Pharmacological Management of Type 2 Diabetes (2007) and Joslin Diabetes Center & Joslin Clinic, Clinical Guideline for Adults with Diabetes (2008). The guidelines may specify a desired biomarker value, e.g., a fasting blood glucose value of less than 100 mg/dl, or the clinician can specify a desired biomarker value based on the clinician's training and experience in treating patients with diabetes. However, such guidelines do not specify biomarker collection procedures for parameter adjustments to support specific therapies used in optimizing a diabetic patient's therapy. Subsequently, diabetic patients often must measure their glucose levels with little structure for collection and with little regard to lifestyle factors. Such unstructured collection of glucose levels can result in some biomarker measurements lacking interpretative context, thereby reducing the value of such measurements to clinicians and other health care providers. Thus, there is a need to provide structured collection procedures for diagnostic or therapy support of a patient with diabetes or other chronic diseases.

Patients with diabetes and their healthcare professionals interact with a variety of medical devices and systems to help manage the disease, including performing structured collection procedures. For each of these differing types of medical devices, there is a need to aggregate, manipulate, manage, present, and communicate diagnostic data and prescriptive data from multiple data sources in an efficient manner to improve the care and health of a person with diabetes, so the person with diabetes can lead a full life and reduce the risk of complications from diabetes.

When designing an overall system for diabetes management or an application residing on a given medical device in the system, there is a further need to identify and implement extension points in the system to support future growth.

This section provides background information related to the present disclosure which is not necessarily prior art.

### SUMMARY

This section provides a general summary of the disclosure, and is not a comprehensive disclosure of its full scope or all of its features.

A mobile device for managing diabetes care of a patent is disclosed. The device has a data entry interface configured to receive blood glucose measures for the patient and store the glucose measures in a patient log residing on the device. The device has a selection module that operates to selectively analyze the glucose measure in the patient log and select a given structural collection procedure from a plurality of standardized collection procedures.

According to an alternate teaching, the mobile device as disclosed above further has an administrative module in data communication with the data store. The administrative module prompts the patient, via the display device, to input glucose measures into the data entry interface.

According to an alternate teaching, any of the above systems further has a wireless transceiver configured to transmit lifestyle data through a telephone network to a medical service provider. A data analysis module operates selectively to analyze the glucose measures in the patient log and interfaces with the wireless transceiver to transmit the blood glucose levels to a medical service provider when one of a predetermined amount of time or a predetermined number of blood glucose samples are taken.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 represents a system for managing blood glucose levels according to the present teachings;

FIG. 2 represents a mobile computing device according to the present disclosure;

FIG. 3 represents a diagram of system modules defined within a cellphone according to the present teachings;

FIGS. 4A and 4B represent flowcharts of structured testing protocols in predetermined routines according to the present teachings;

FIGS. 5A-5C represent screen data from the system of the present teachings;

FIG. 6 represents a flowchart describing an exemplar decision process regarding the application of structured testing protocols;

FIG. 7 is a diagram representing the interaction of components within the system.

FIG. 8 represents a system diagram according to the present teachings;

FIGS. 9A-9H represent screen shots of data entry modules according to the present teachings;

FIGS. 10A-10D represent screen shots of the home screen according to the present teachings;

FIGS. 11A-11F represent data input screens according to the present teachings;

FIGS. 12A-12C represent views of the logbook on the mobile device;

FIGS. 13A-13D represent views of a data collection module for a three-day test procedure;

FIGS. 14A-14C represent a report for the results of a three-day test;

FIGS. 15A-15B represent a display of graphs for the results of a three-day testing protocol;

FIGS. 16A-16D represent statistics for the results of a three-day testing protocol;

FIGS. 17A-17B represent tabular views of a report for a three-day testing protocol;

FIGS. 18A and 18B represent a testing in pair test procedure;

FIGS. 19A-19H represent images of a testing in pair protocol;

FIGS. 20A-20C represent reporting of a testing in pair protocol;

FIG. 21 represents a report sharing screen according to the present teachings;

FIGS. 22A-22H represent optional reports on the mobile device;

FIG. 23 represents a sharing by email screen according to the present teachings;

FIG. 24 represents a settings screen for the setting module;

FIGS. 25A-25G represent screens related to reminders;

FIG. 26 represents screen for setting data sharing parameters; and

Corresponding reference numerals indicate corresponding parts throughout the several views of the drawings.

### DETAILED DESCRIPTION

Example embodiments will now be described more fully with reference to the accompanying drawings.

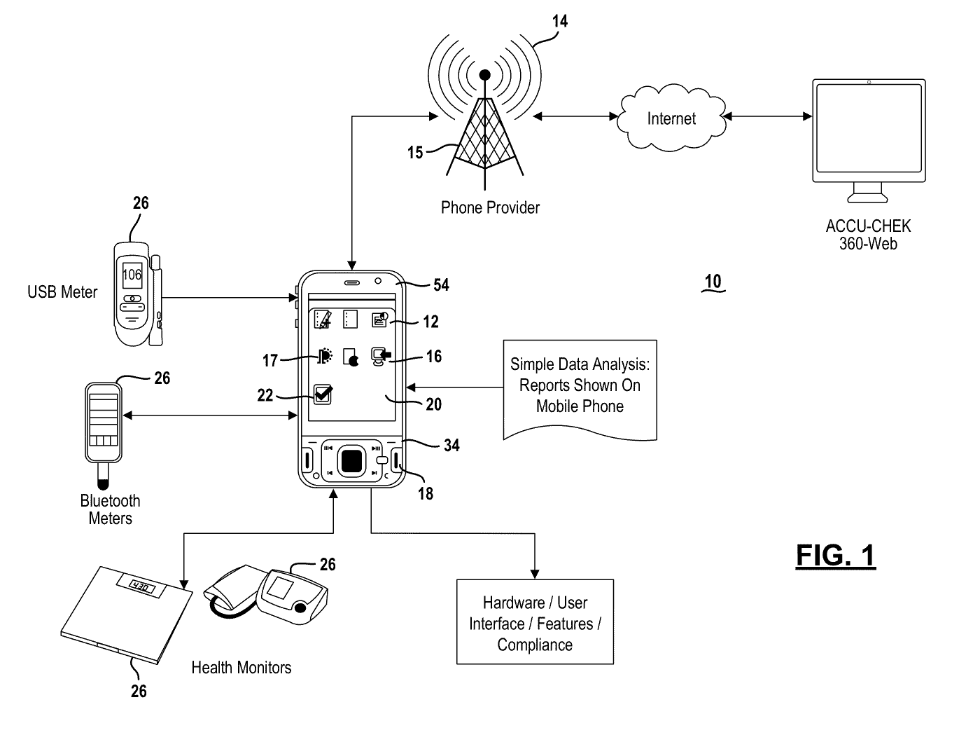

As shown generally in FIGS. 1-6, the system for managing diabetes care 10 of a patient according to the present teachings provides a mobile phone or computing device 12 capable of allowing manual entry and storage of user medical information. The system for managing diabetes care 10 of a patient also provides for a communications system 15 to allow the transfer of medical data through a telephone network 14 to a medical source provider via email or messaging protocol.

A first instruction set 16 within the mobile computing device 12 functions to support functionality not directly associated with the wireless communication of medical data, while a second instruction set 17 within the mobile device functions to support the functionality related to communicating the medical data through the communications system 15. The first instruction set 16 supports functionality which can be, for instance, data entry, reminders, the prompting of structured test procedures, and simple reporting through a display screen 20. As described further below, when network access is available, medical data can be uploaded to medical service providers such as caregivers and HCP's.

The mobile computing device 12 through the second instruction set 17 is configured to communicate medical data through mobile environments such as a wireless telephone system or hybrid-web local application to transfer the medical data. Optionally, the communications system 15 can transfer the data using protocols AC360 DMS web and blue tooth protocol. In addition to the manual entry of data, other devices 26 can automatically update medical information into the mobile computing device 12. These devices 26, for example, include blue tooth enabled BG meters, heart rate monitors and blood pressure cuffs, and weight scales. Optionally, additional non-patient medical information from cloud based databases can be uploaded to the mobile computing device 12. This information can be updated and incorporated through the wireless system into the mobile computing device 12 as a software update. These datasets can, for example, include insulin dosing advice, additional structured protocols, and food database information.

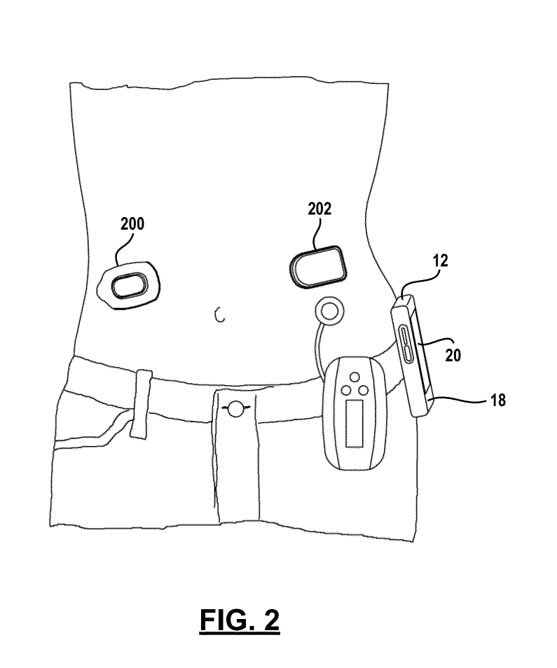

As shown in FIGS. 1 and 2, the mobile computing device 12 has a data input mechanism 18 such as a keyboard. As is known, the data input mechanism 18 can be integrated into the display screen 20. Optionally, the display screen 20 will prompt the user to input medical factors 22 such as blood glucose, blood pressure, carbohydrate energy units and weight. The user has the ability to vary the value of the units for the samples being inputted. For example, blood glucose can be inputted in mg/dL or mmo/L.

At various times, as described in detail below, the mobile computing device 12 will prompt the user to input bG target values and ranges. These can either be a single bG target range or dual pre-prandial and post-prandial range along with a hypoglycemic limit The mobile computing device 12 can supply default bG target ranges which can vary based on a geographic region of the world. In addition to prompting a user for biological measurements, the mobile computing device 12 allows for the user to input other information which may be useful to a treating medical professional. This information can for example include a medications list, an exercise schedule, and a physiological state. The mobile computing device 12, through the display, can additionally allow the user to configure the mechanism so that reminders can occur. For instance, audible or vibrating reminders can occur. Additionally, the user can update the desired bG value. The inputted value should be checked to determine if it is within an acceptable level.

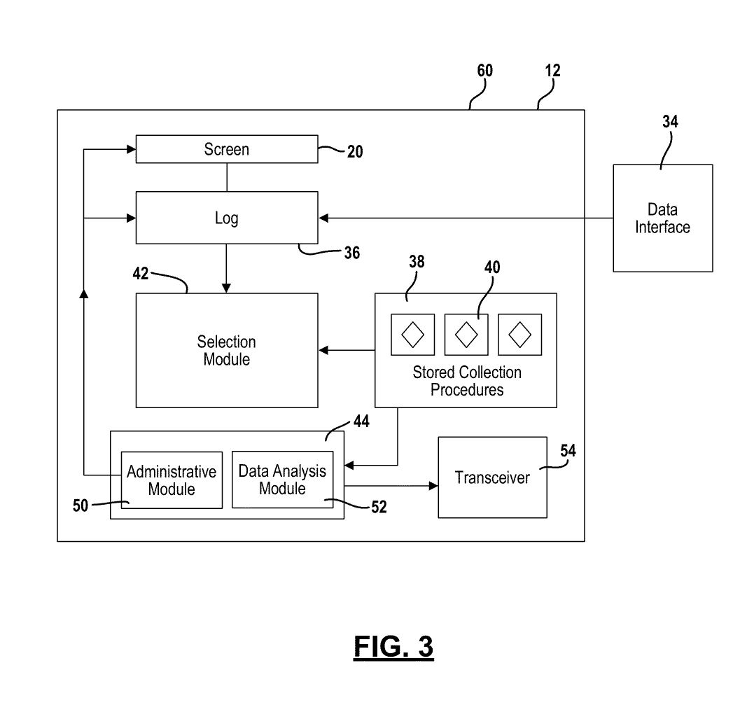

As best seen in FIG. 3, a representative mobile computing device 12 has a display screen 20, data entry interface 34 and log 36. The data entry interface 34 is configured to receive glucose measures for the patient and store the glucose measures in the log 36. An associated data store 38 stores a plurality of structured collection procedures 40. As described below, each structured collection procedure 40 specifies one or more collection events for obtaining glucose measures from the patient.

The mobile computing device 12 further has a selection module 42 that operates selectively to analyze the medical data such as glucose measures in the log 36 and select and present to the user, through alarms and prompting, a given structured collection procedure 40. An administrative module 50 in data communication with the data store 38 prompts the patient to input glucose measures into the data entry interface in accordance with the structured collection procedure 40. A wireless transceiver 54 is coupled to an administrative module 50 and is configured to transmit data from the log 36 to a health care professional through the wireless telephone network 14.

A data analysis module 52 associated with the administrative module 50 selectively operates to analyze the glucose measures in the log 36 and interface with the transceiver 54 to transmit the data from the log to a health care professional when the medical data is outside of a predetermined window. Additionally, medical data can be transmitted at predetermined time intervals, when a predetermined number of samples are collected or as requested by a treating physician. Upon detecting certain events, the administrative module 50 may prompt a patient to engage in a more rigorous structured collection procedure 40 or may request further data related to life style events. This may occur if, for instance, the blood glucose level detected in the log 36 is trending in an undesirable direction or if it determined that the caloric intake for the patient is improper for the patient's weight.

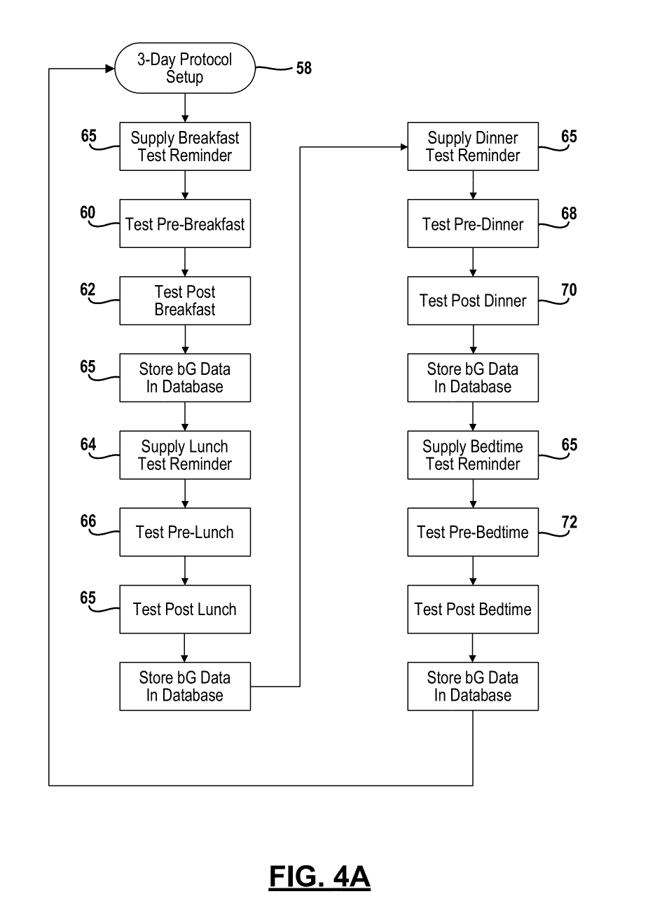

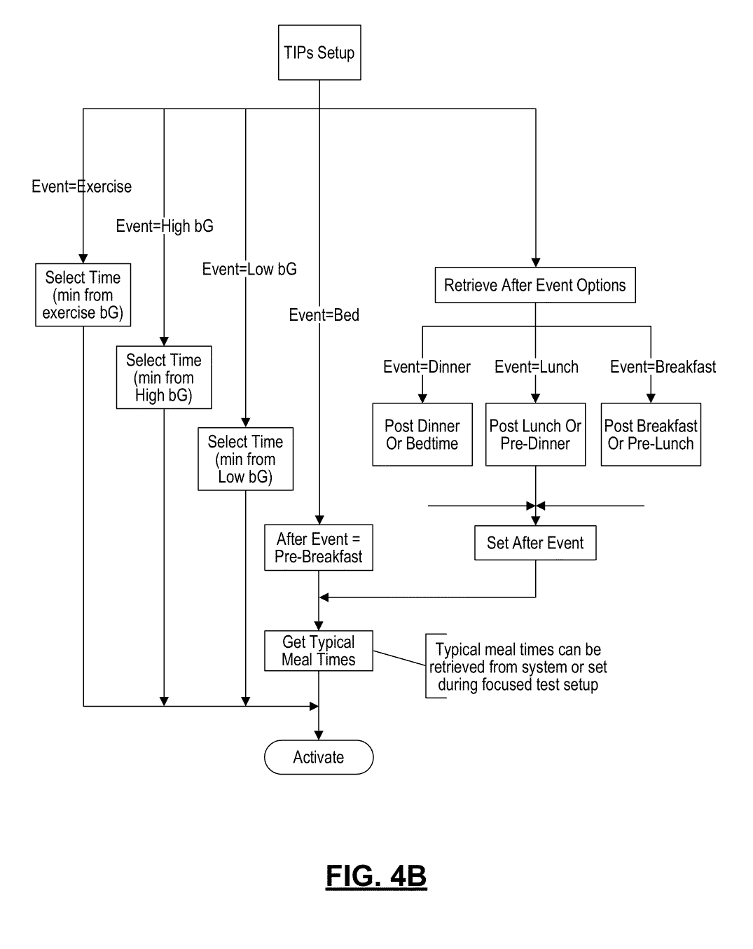

As is shown in FIGS. 4A and 4B, the mobile computing device 12 allows for the manual or automatic prompting of one of several structured testing regimes or procedures 40 as required by a treating physician. Additionally, an indication that a structured testing regime is running as well as its progress can be visible on the display screen 20. It is envisioned that at least two structured collection procedures 40 can be available. As shown in FIG. 4A, the first is a three-day structured test. The user can be prompted to set variables for the test such as the start date, breakfast, lunch, dinner and bed times in process block 58. An optional alarm or reminder for required bG test time can be set before the structured test actually starts. During the three-day structured test, the mobile computing device 12 will prompt the user at each meal to enter information related to the meal as well as energy levels. The user will be notified through the display device when an extension of the test regime is required due to incomplete bG measurement data.

The three-day structured test involves the patient checking his/her bG several times during consecutive twenty-four hour periods and manually recording the data into the mobile computing device 12. The mobile computing device 12 then stores the data in the log 36. Preferably, the three-day bG test is done with the patient checking his/her bG levels at seven different times during the day: 1) pre-breakfast 60; 2) post-breakfast 62; 3) pre-lunch 66; 4) post-lunch 65; 5) pre-dinner 68; 6) post-dinner 70; and 7) bedtime 72. The results of each of the bG tests may be recorded manually by the patient. Optionally, a blue tooth-enabled meter 26 can transfer the data to the mobile computing device 12. The bG tests need to be performed within the context for each of the above-described seven events (which may include a predetermined time window). As it is important that the patient record all of the obtained bG information correctly on the three-day bG test chart, the mobile computing device 12 can prompt the patient to take the appropriate test with an alarm.

In configuring the three-day test protocol 58, the user can input the testing start date, breakfast, lunch, dinner times and bed time. Optionally, the user will receive reminders 64 which correspond to each meal or bed time. The user can configure the reminders related to data to be logged. Optionally, when a reminder 64 is associated with data in a data field, the user will be required to pick from the list. For example, should the reminder be associated with insulin, the user will be required to pick from a list of insulins.

While configuring a three-day profile test, the user has the option to set a reminder 64 prior to the initiation of the tests. While running a three-day profile, the system determines if the last entry is the last entry of the day, or of the test regime. Should the entry be the last, the system stops the automatic alarm system. The system will confirm the data entries for the calendar are complete when five bG entries have been stored within a twenty-four hour period. This is opposed to relying on calendar days for scheduling. At the end of one of the structured collection procedure 40, the system will allow the user to ask the medical provider questions such as “What can I do next?” or “What questions do I have for my health care provider?”

Should the user or the system require a testing-in-pairs structured test (see FIG. 4B), the system will prompt the user to collect bG data before and after a selected event for seven consecutive occurrences. These occurrences can be, for example, breakfast, lunch, dinner, bed time, exercises. The times for the samples is also entered.

This test involves having an individual obtain pairs of bG values before and after various events. For example, an individual can obtain a bG value before a specific meal, for example before lunch, and another bG value within a specified time after the lunch meal. The “before” and “after” bG values form a related “pair” of bG values and can be used as data for a “Testing In Pairs” (TIPs) test. Collecting and reviewing a plurality of related pairs of before/after bG test data for various events throughout the day (e.g., breakfast, lunch, dinner), while considering the type of food that was consumed at each meal, may help give the individual a better idea of how his/her bG levels are affected by certain foods or events, and thus may help the individual to better manage her/his bG levels throughout the day.

The paired bG values can then be manually or automatically recorded into the system by the user 80. Moreover, the individual must be attentive to the time periods during which the “before” and “after” bG test values must be obtained. Missing a “before” meal bG test will prevent the use of an “after” meal bG test result, for the purpose of constructing a “pair” of bG values for the test.

In configuring a testing in pairs structured collection procedures 40, in the mobile computing device 12, the user is prompted to test their bG before and after a selected event for seven consecutive occurrences. The user can configure the test by selecting from a list of events. Optionally, this list can include the standard TIP types of inputs such as breakfast, lunch, dinner, bedtime, exercise list values, high bG and low bG as well as custom types of hyperglycemic bG value, hypoglycemic bG value, insulin list values, blood pressure and energy level.

Data from either of the above described tests is stored within the log 36 either on the mobile computing device 12 or at a controlled location accessible through a wireless network. Additionally, examples of data which can be stored in the log 36 include blood glucose, blood pressure, meals/snacks, energy levels, exercise, medication, weight and additional notes such as physiological conditions.

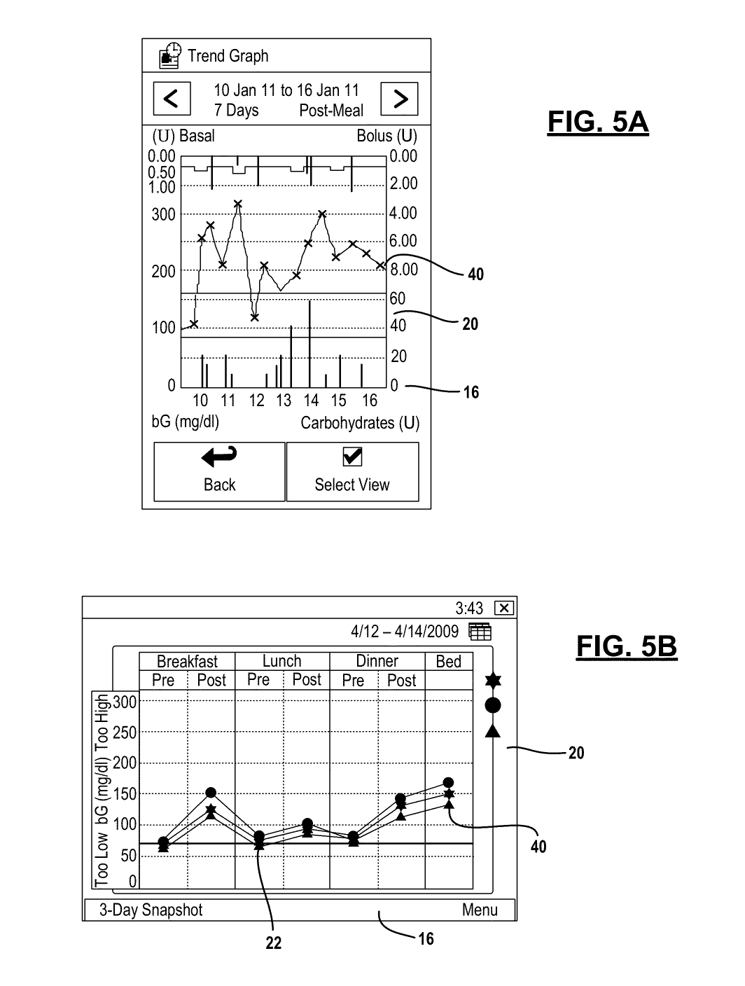

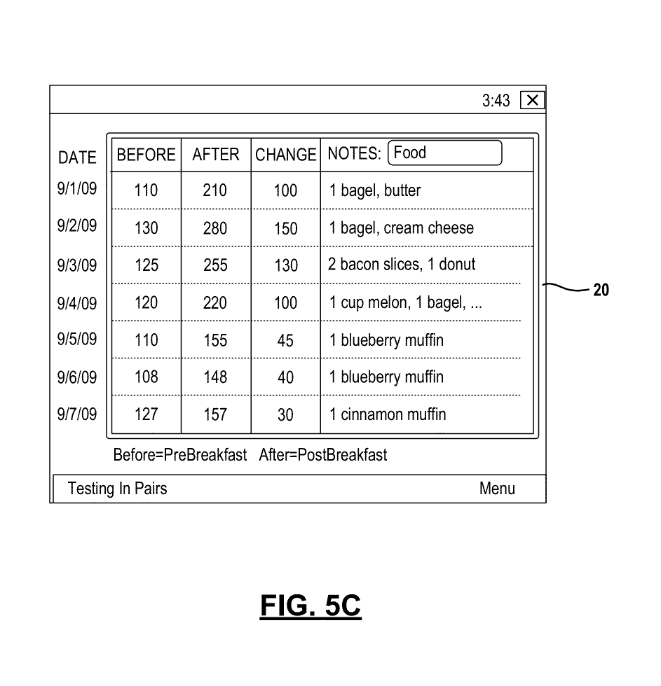

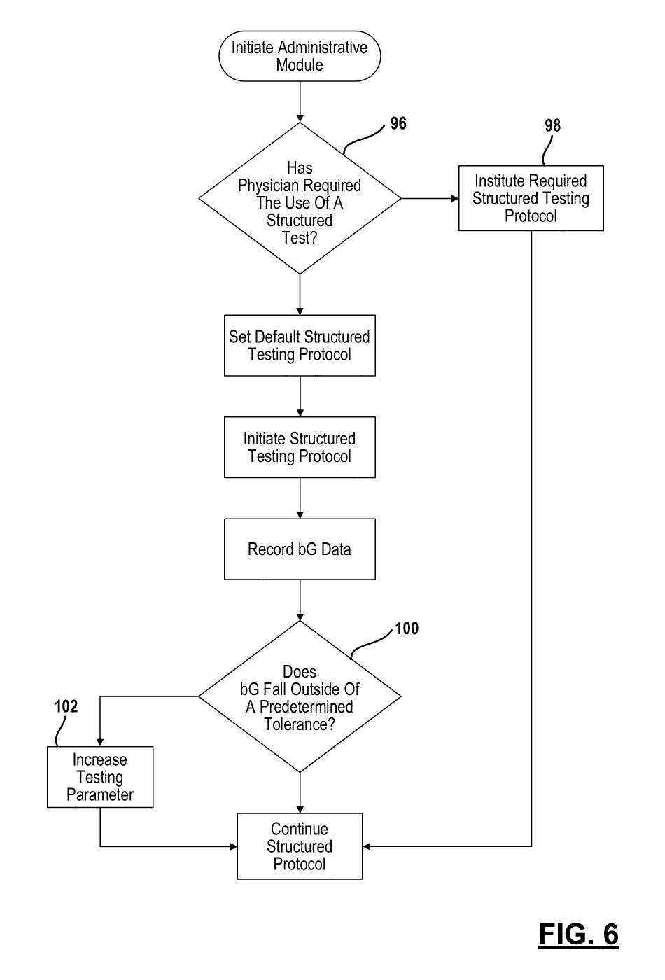

The mobile computing device 12 allows for a user to view the results in the form of graphs and reports. The mobile computing device 12 allows the user to view statistics such as a bG trend report, three-day test report and testing in pair report. An example of these reports is shown in FIGS. 5A-5C. The bG trend graphs or statistics for two day, seven day, and thirty days are available. The system can limit the display to cover date ranges only, covering the entire amount of data stored. Optionally, only data related to a specific structured test will be displayed.

In a tabular view, the system will report information related to the number of tests; average number of tests per day; average bG and standard deviation; average pre-meal and post-meal bG values; total number and percentage of bG values within the bG target range; total number and percentage of bG values above the bG target range; total number and percentage of bG values below the bG target range but above the hypoglycemic target value; total number and percentage of bG values equal to or less than the hypoglycemic target value; average daily total calories per day; and average daily total carbohydrates per day. While displaying the report, statistics can be calculated such as those defined in the “Standard Business Rules for Diabetes Data Management Standard.” The output of the report can mirror the format of reports generated by known bG meters. As shown in FIGS. 5A and 5B, graphs can be viewable in either portrait or in landscape, allowing panning and zooming of the view of the graph. Entered and calculated data can be displayed.

In configuring the mobile computing device 12, the user can set data units for the input of collected data. The system will have predefined units associated therewith. The user can optionally set target bG levels with the advice of a treating physician. Additionally, the user can set an insulin list as well as a medication and exercise list. The user also can optionally configure the second instruction set 17. In this regard, the user can configure automatic SMS messaging. This includes a selected list of recipients with corresponding telephone numbers. The user can be notified once an SMS, MMS or email message has been sent.

Additionally programmable is a log of reminders64. In this regard, reminders 64 can include start date/time as well as recurrence of none, daily, weekly, monthly or selected days of the week. Optionally, the user can set the sound and volume of a reminder alert. The log of reminders 64 will be stored by name, allowing the user to retrieve them quickly. When a reminder occurs, the user can accept the reminder, snooze it, or dismiss (cancel) it. When the reminder is accepted, the user can be prompted to enter data. Optionally, when data has been entered with a predetermined time period of a schedules time, the reminder will be cancelled.

As described above, the mobile computing device 12 contains a plurality of predefined structured collection procedures 40. The user can configure these structured tests. Optionally, the user can start and stop the structured collection procedure 40. The user should be able to identify by looking at the mobile device display that a structured test is running, and optionally its progress. The system can optionally allow only one structured test to be running at a time.

While the structured test is running, the system will prompt the user to perform bG measurements for the structured test at the configured times. The user can change the default settings to silence the reminders. When a user initiates a structured test which has been previously run, the times set for the last test will be revised. The mobile computing device 12 will indicate to the user when the structured test is complete.

Users will be reminded to input meal information, bG and energy level. The user will be notified if an extension of the test regime is required due to missing bG data. In this regard, when test data for a day is incomplete, the user will be able to discard the incomplete day and extend the test.

The user can enter pre-event timing for each event. When the event is a meal, the user can select from post event meal options. Log data can be edited by a user as needed. The user will have the ability to create, edit or delete logbook entries. The user can optionally create a logbook entry consistent with a single bG entry as well as date and time. Additionally, the user can log data as associated entries consisting of any combination of data values along with date and time. To increase the ease of entry, the user can alter the order of data entry.

Optionally, the first instruction set 16 will allow for separate or associated logs for insulin injections, blood glucose, blood pressure, meal/snacks, energy level, exercise, medication and notes. Associated with each can be prescribed entry information such as insulin or medication name, dosage amount. With respect to blood glucose, should a structured collection procedure 40 be running, the user can indicate that they do not have bG data to input. With respect to blood pressure, the mobile computing device 12 will accept systolic and diastolic values as well as date and time. With respect to meals, the system can accept carbohydrate value, energy units or meal size indicators of Small, Medium, or Large along with date and time. For energy level, the user should be able to input values ranging from 1 to 5. Exercise can be recorded as high, medium or low intensity along with date and time.

The user can view the log entries prior to forwarding them to the medical care provider. The log data can be by date and time ranges. As described below, statistical reports are viewable on a graphical user interface on the mobile computing device 12. For example, bG trend graphs can be viewed. A calculate trend line associated with recorded information can be displayed.

The logbook entry can consist of user editable date and time plus one other data value. Optionally, the acceptable date ranges, which can be edited, can be limited. For example, when a record is associated with a structured test, the ability to edit the data field can be subdued. Data entered into the log can be converted into a standardized measure prior to storage in the log. In some circumstances, the user can have the option to take additional tests outside of a structured testing regime. As such, a user can be prompted to determine whether the data should be associated with the structured test or just stored within the log. If the data is being entered when a structured is being conducted, the system will determine if the data is entered within an acceptance window for the structured test. If the data is being entered within the structured test acceptance window, the mobile computing device 12 will automatically associate the entered data with the test.

The mobile computing device 12 can facilitate the backup of data log and user preference information. This backup can be either through the wireless system or through a wire coupled to the mobile computing device 12. It is envisioned the user can adjust factors such as the name and storage location for a backed up data log. Optionally, the user can back up the data file through the email system. The system provides a methodology to automate software updates. Within this functionality is ability for the user to delete the software, and optionally all of its stored data and preface settings. Functionally, the user is allowed to update the software while maintaining their preferences and data logs.

To view the results of structured protocols such as the three-day or testing in pairs protocol, the user is prompted to determine if the results should be presented in tabular or chart format. In addition to the present report data, the result of former tests is also available.

FIG. 6 represents a flowchart showing an optional control logic for the administrative module 50. This administrative module 50 functions to designate which of the several structured collection procedures 40 will be presented to the user. Optionally, the mobile computing device 12 will query the user to determine if a treating medical service provider has required a specific structured test be used 96. If a specific structured collection procedure 40 has been assigned, the system 10 will use the specified test 98. The user will then be presented with an option to specify which structured test they would like to use.

The administrative module 50 can optionally monitor the medical information inputted by the user 100. Should the value of the data fall outside of a set of predetermined values, the administrative module 50 can take several intermittent but escalating steps to gather more information 102. These steps may be initiated if, for example, bG level is trending toward an unacceptable level, or if, for instance, caloric intake is trending into an unsafe level without a corresponding change in exercise. In these scenarios, the administrative module 50 can prompt a user to input more information related to diet. Additionally, the administration module 50 can request the user initiate one of the structures test scenarios. Medical information can further be forwarded via the communications system 15 to the treating medical professional.

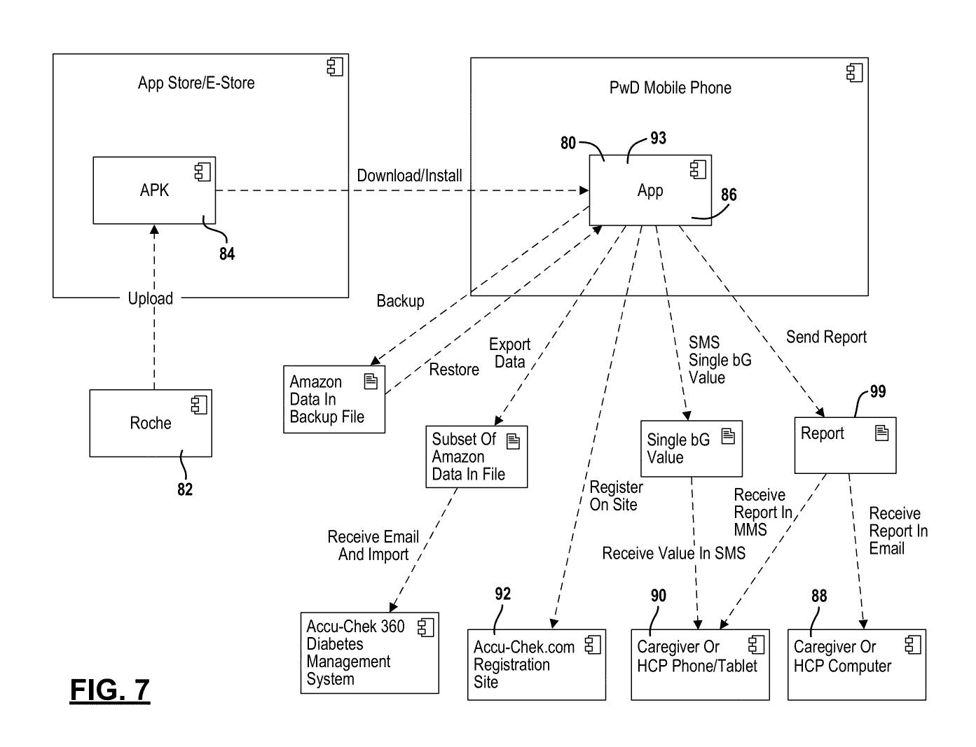

As shown in FIG. 7, software 84 is downloaded into a mobile computing device 12. Registration information from the software 84 is forwarded to a medical software provider 92. As described above, medical data can be forwarded via email, MMS, SMS, HTTP or email to an analyst, a caregiver's computer 88, or a caregiver's mobile device 90. This includes a mechanism for registering a user's name and information related to mobile computing device 12. This can, for example, include a phone number associated with the mobile device, and should the mobile computing device be a bG meter, serial number information can be registered. In registering the user, the system will collect information related to the diabetes type as well as therapy type. The therapy type can include diet and exercise only, oral medication, insulin injections or insulin pump. When complete, the registration information will be emailed or transmitted via MMS or SMS to a specified location.

The mobile computing device 12 has a module 93 which allows for the configuration of SMS, MMS, HTTP or email transmission of information stored within the log. The module 93 is configured to allow the user to augment a list of recipients of the log information. Optionally, a code which functions as a pointer can be provided to the user. This code facilitates the automatic updating of several recipient numbers. The module 93 can support the removal or editing of a recipient's information. Optionally, the module can notify the user when data is transmitted.

As seen, the reports 99 can be sent to the physician's computing device. In this regard, the report can be sent to the physician's phone or computer 88. Additionally, the report can be sent to a data analysis system 86 to track medical information related thereto.

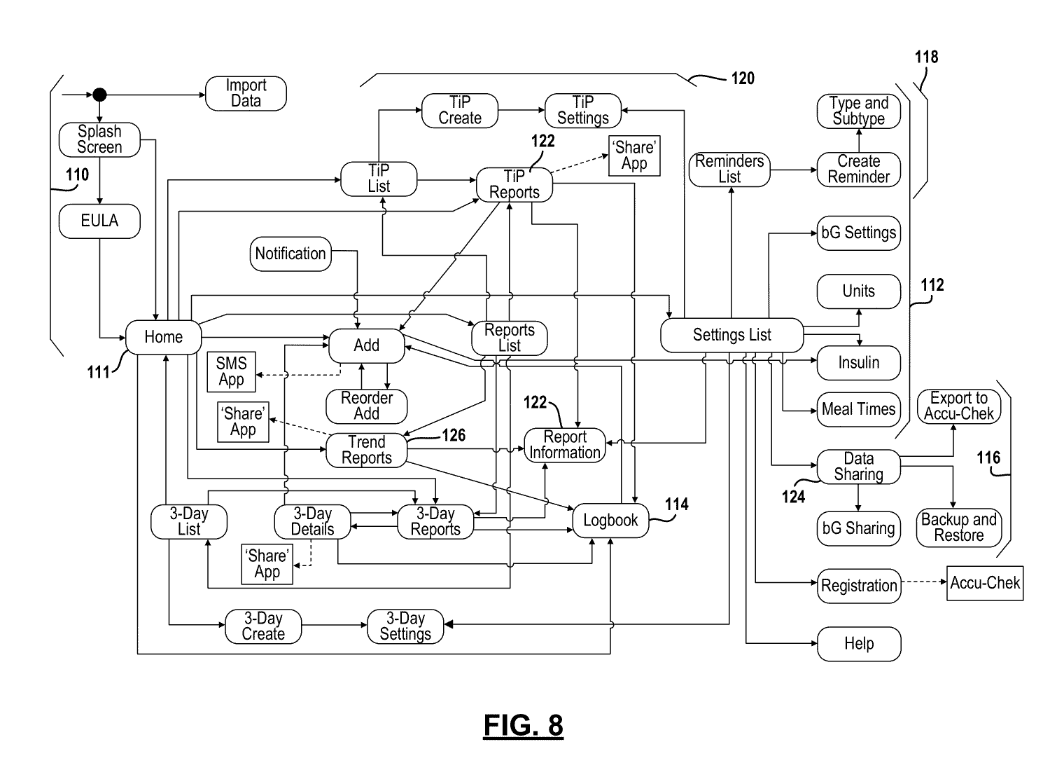

As shown in FIG. 8, the system for managing insulin levels in a patient can have an install module 110, a setting module 112, a logbook 114, a data export module 116, a reminder module 118, a structured testing module 120, a reporting module 122, a communications module 124, and an analytics module 126. The system functions to shepherd the installation of the software on to the mobile device using the install module 110. Optionally, the install module will interface with a home computer to automatically install updates to the software or pre-set variables or setting. From the install module 110, the system allows the home screen 111 to function as a central point within the software.

From the home screen 111, the user is prompted to go to the setting module 112. When started, the setting module 112 prompts or allows the user to set parameters such as bG levels, units, insulin levels and meal times. The setting module 112 also allows the user to set data sharing parameters in the data export module 116. These parameters can include which people or systems health information should be shared with. Also, other data sharing parameters can be, for instance, backup and restore setting or settings for data management systems such as the Accu-Chek® 360° Diabetes Management System.

The setting module 112 also allows the user to set a reminders list in the reminder module 118. As described above, the reminders can include timing for insulin shots, bG measurements or exercise. The home screen 111 allows the user to initiate the report module 122. The report module 122 allows the user to view trend reports for the various testing procedures and health parameters. From the reports module 122, the user can initiate the logbook 114. Optionally, the logbook 114 can be directionally initiated from the home screen 111.

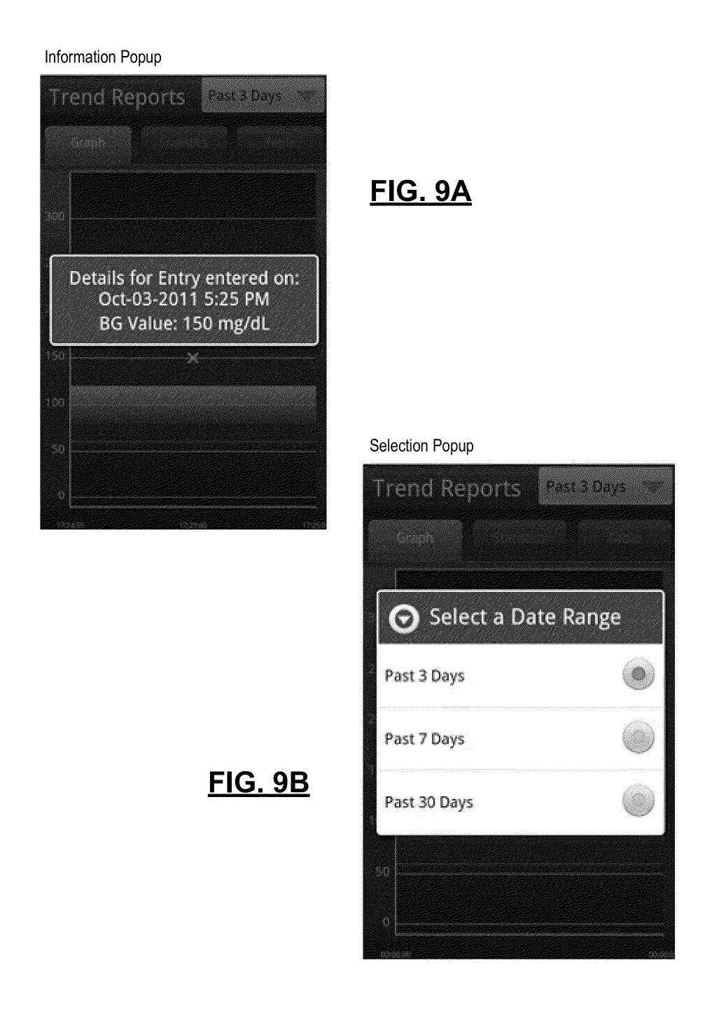

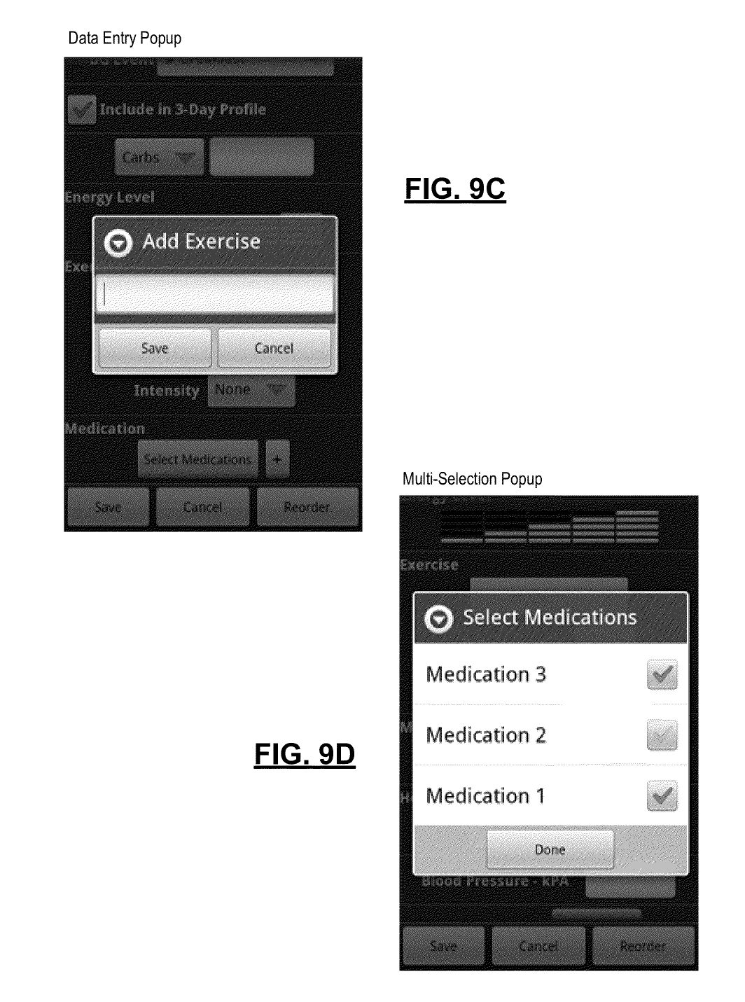

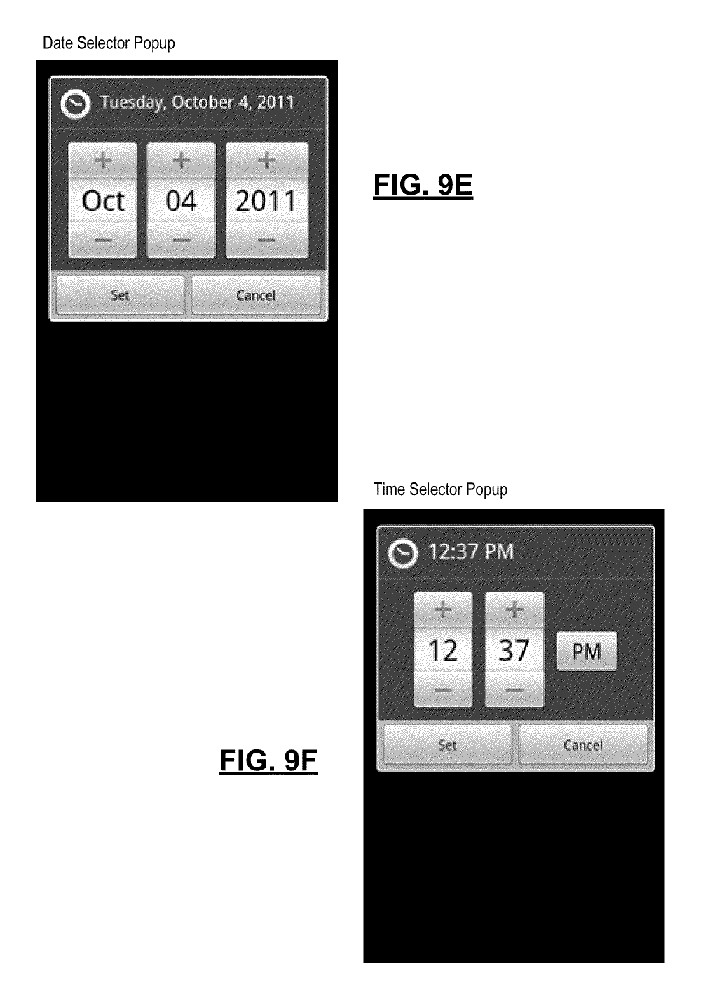

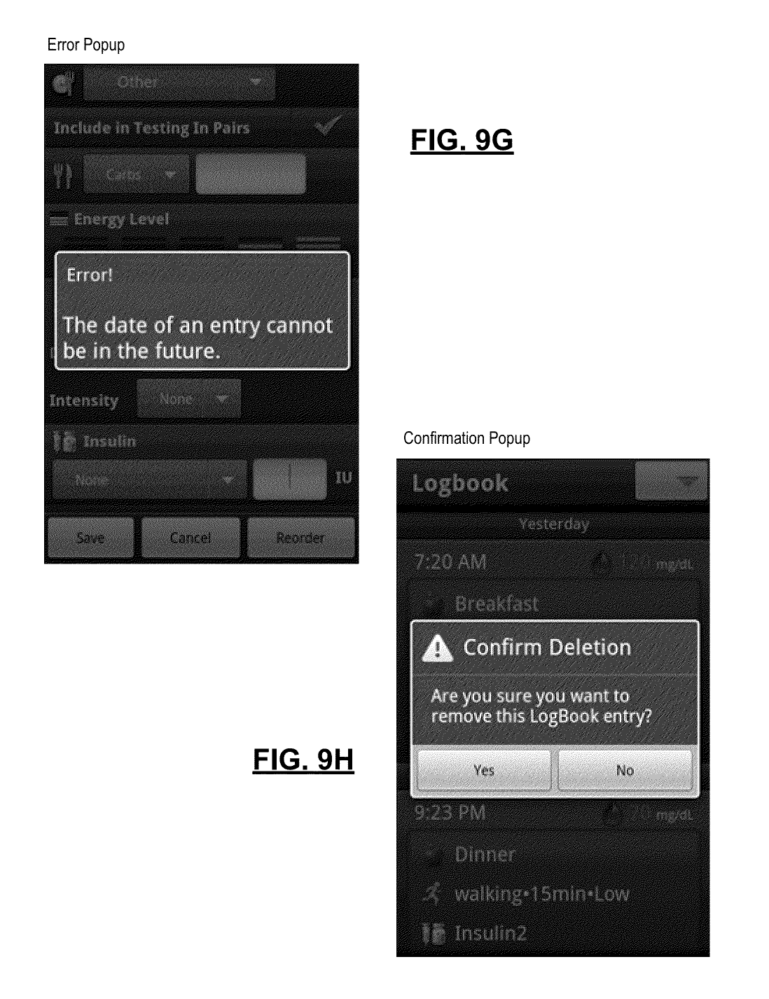

FIGS. 9A-9H represent screen shots of data entry modules according to the present teachings. As can be seen, the system uses pop-up windows to allow the user to input data. The pop-ups can either display specific information (see FIG. 9A), or can act as a location to enter information (FIGS. 9B-9F). This data can include data ranges, exercise, medications, date or time. The system can also use pop-ups to communicate an error condition for data entered or to confirm changes in the data (FIG. 9G or 9H).

The settings module 112 allows a user to input various types of data such as meal time and type information, blood pressure (kPA, mmHg), weight (Kg, lbs), insulin, insulin type (by manufacturer or mix), the amount of exercise, medication, intensity. Additional information can be directed to specific testing protocols. For instance, for a testing in pairs protocol, the system can record breakfast, lunch, dinner, high bG, low bG, bedtime, exercise list and insulin list. Meal information can be entered as carbohydrates, calories or meal size. Information related to trends can relate to time such as 3, 7 or 30 days.

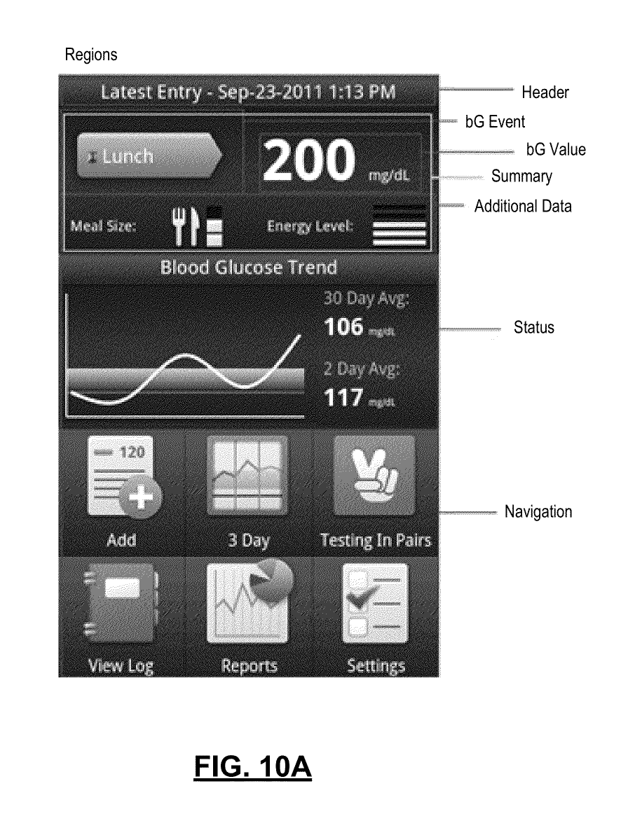

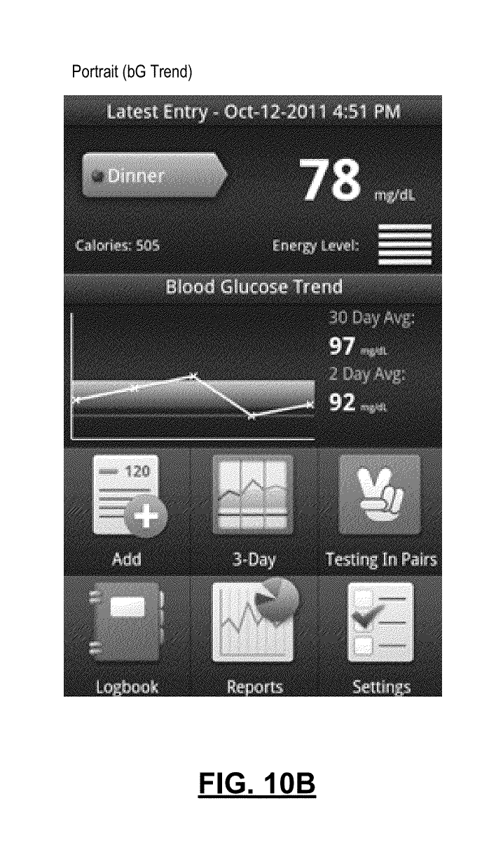

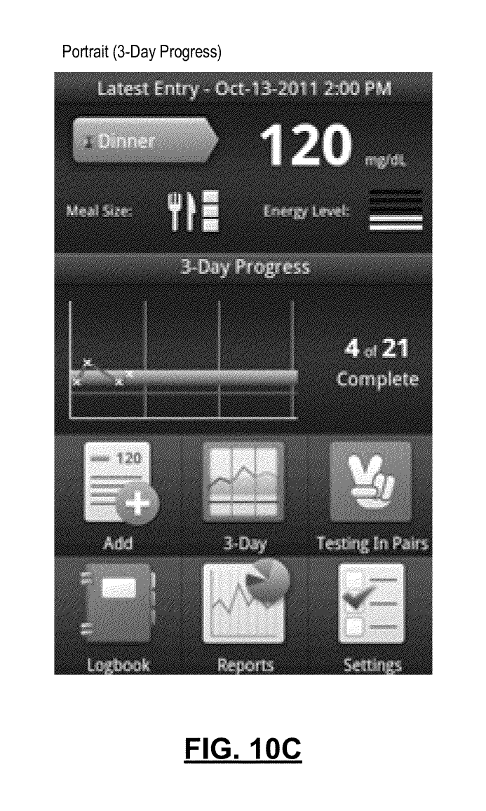

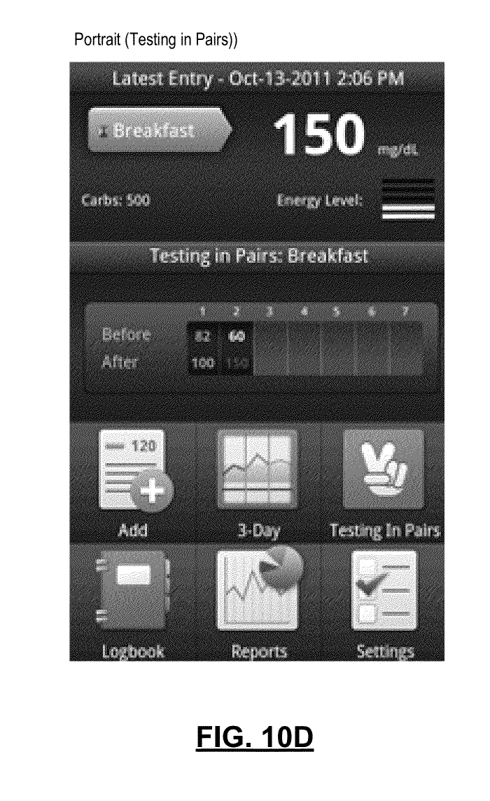

FIGS. 10A-10D represent screen shots of optional home pages from a mobile device according to the present teachings. As shown, the home page can be configured to contain useful information for the user. The user can view trend graphs of, for instance, bG, calories, or the amount of required testing. Trend graphs can be directed to a health parameter such as blood pressure or can be directed to information for a specific testing protocol. Also, optionally, the home page shows links to allow the user to get to the setting module 112, logbook 114, data export module 116, reminder module 118, structured testing module 120, reporting module 122, communications module 124 or analytics module 126.

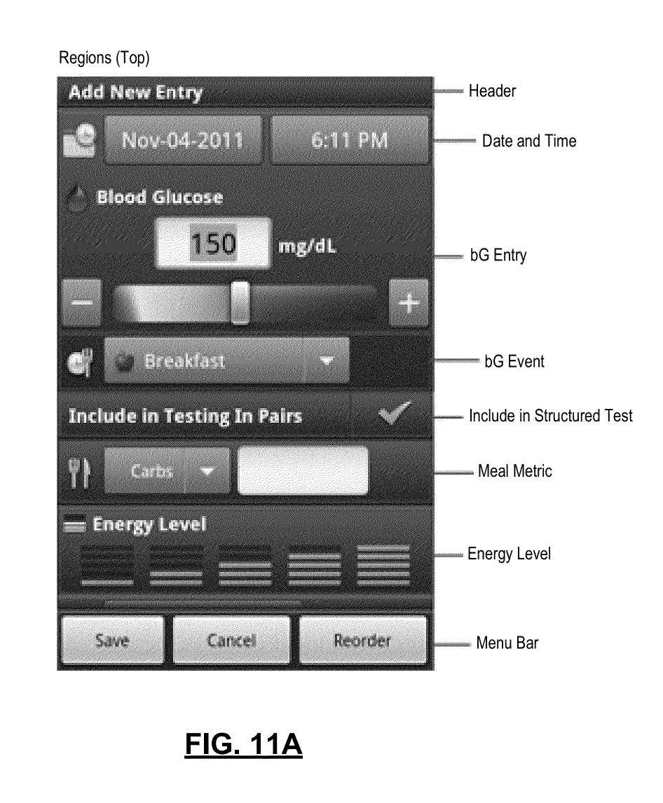

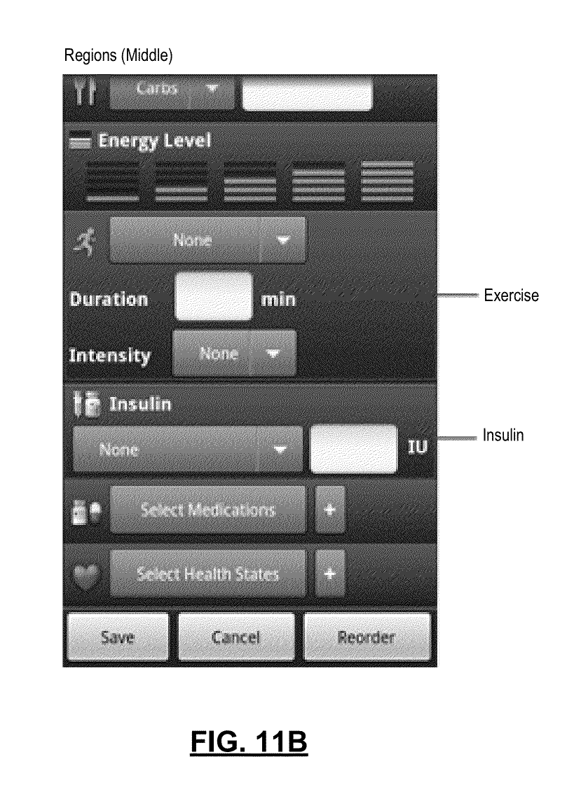

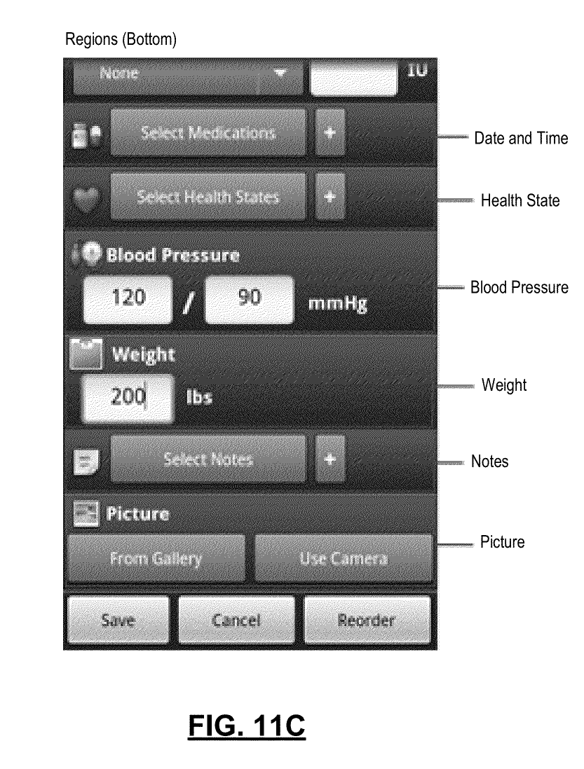

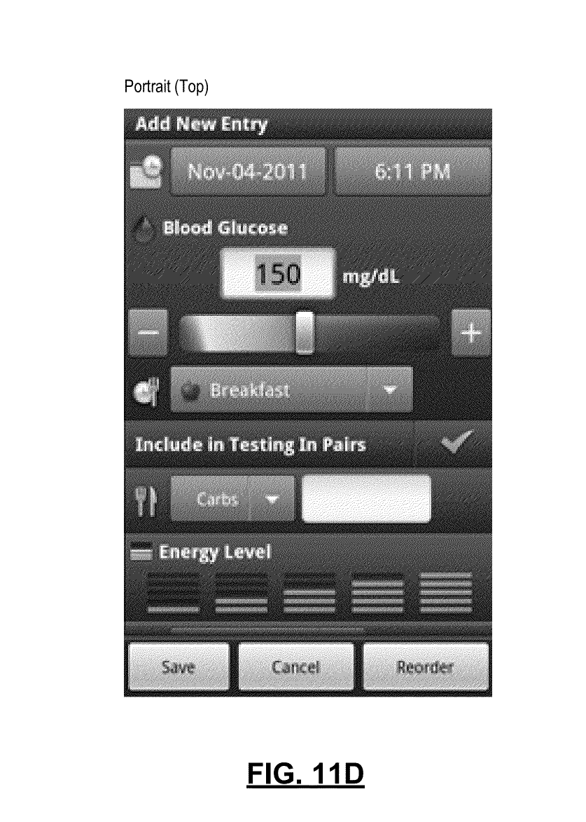

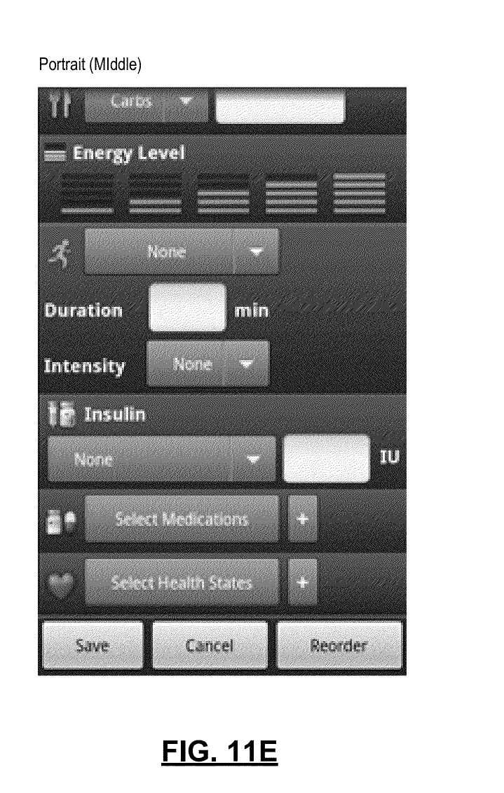

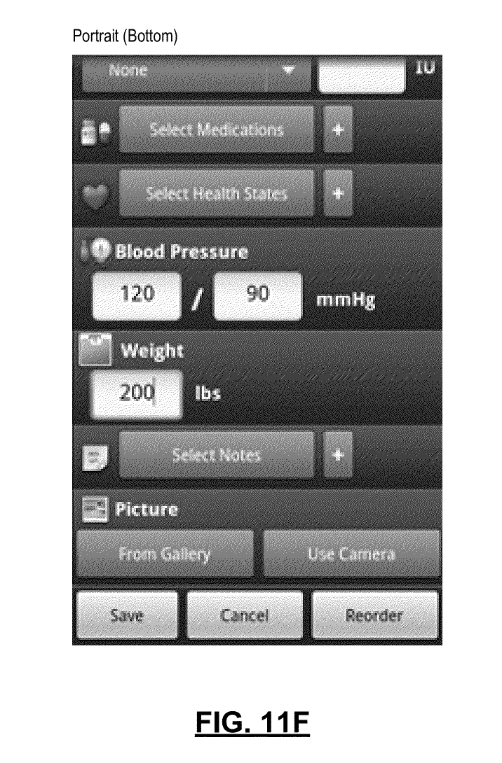

FIGS. 11A-11F represent data input screens according to the present teachings. Shown are screens which allow the user to input information related to bG and amount of carbohydrates consumed for a testing in pairs protocol. Also shown is a screen which will allow the user to input exercise and insulin information. Additional information can be input. This includes energy level, amount and type of exercise, the intensity of exercise, medication taken, blood pressure or health parameters, as well as notes.

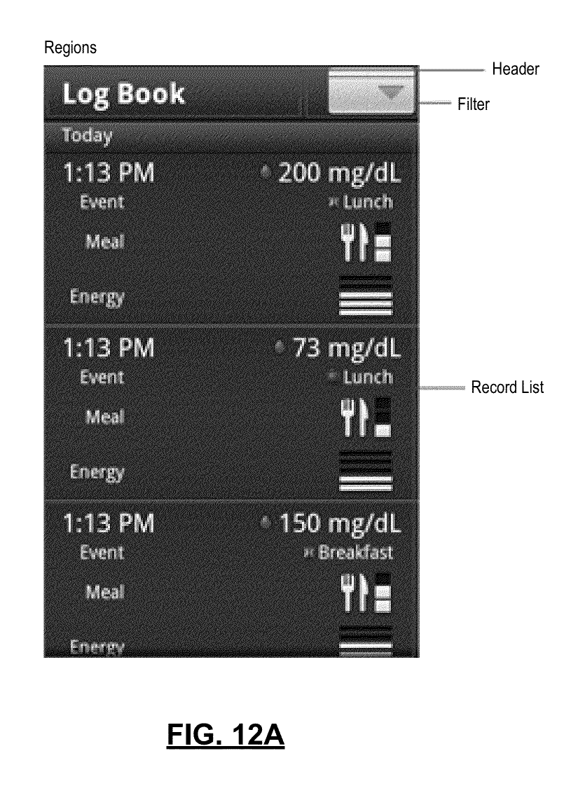

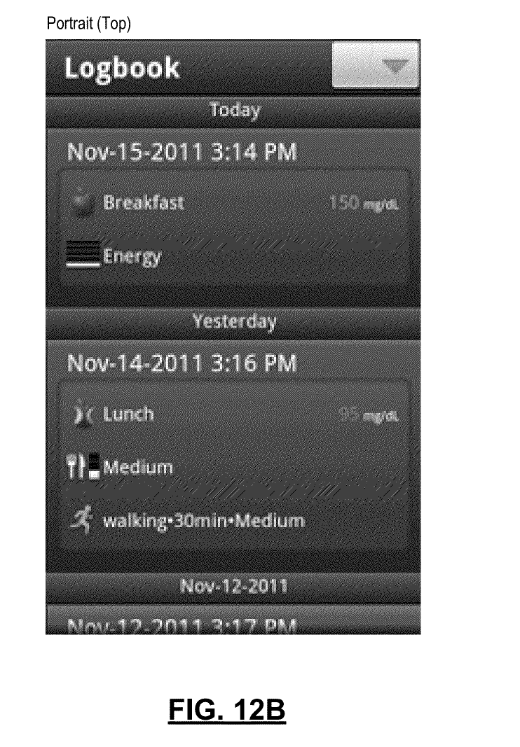

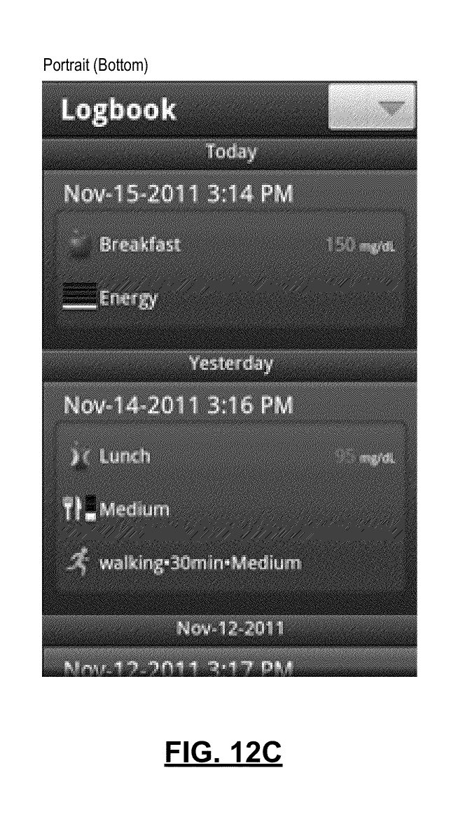

FIGS. 12A-12C represent views of the logbook stored in the mobile device. Shown is health information along with date and time for the particular event. This data can be used by the analysis module to determine which testing protocol is best. The data depicted may be the data shown in the reports or data that is transferred to the physician.

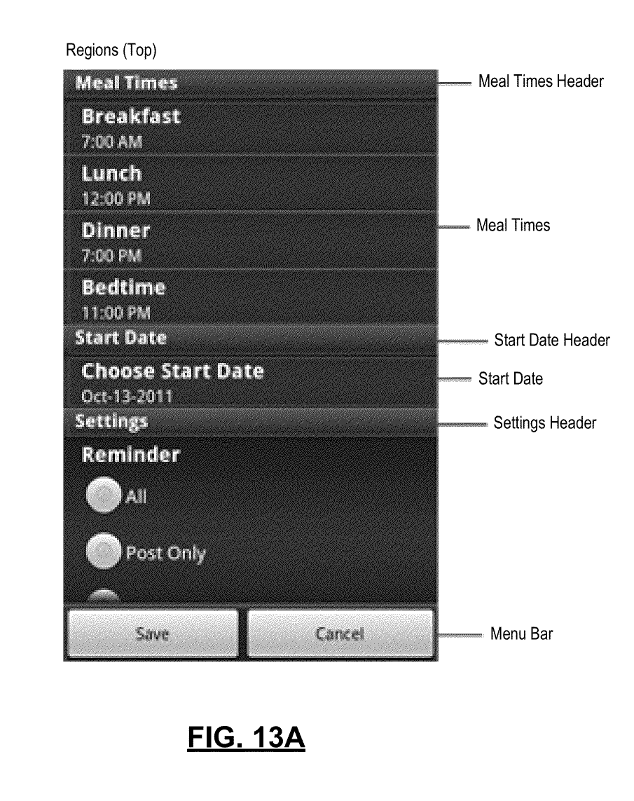

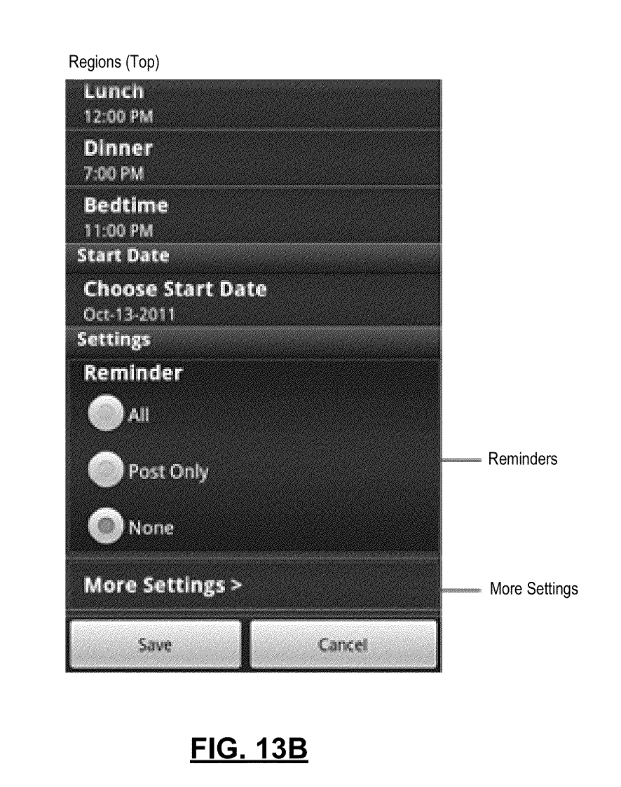

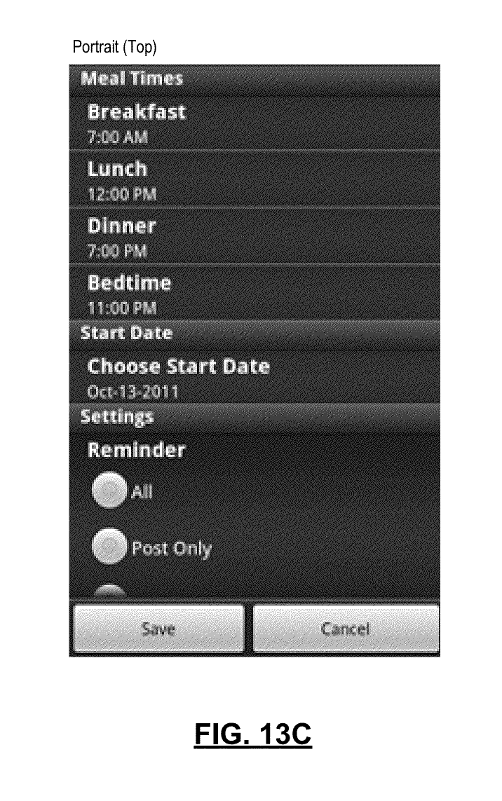

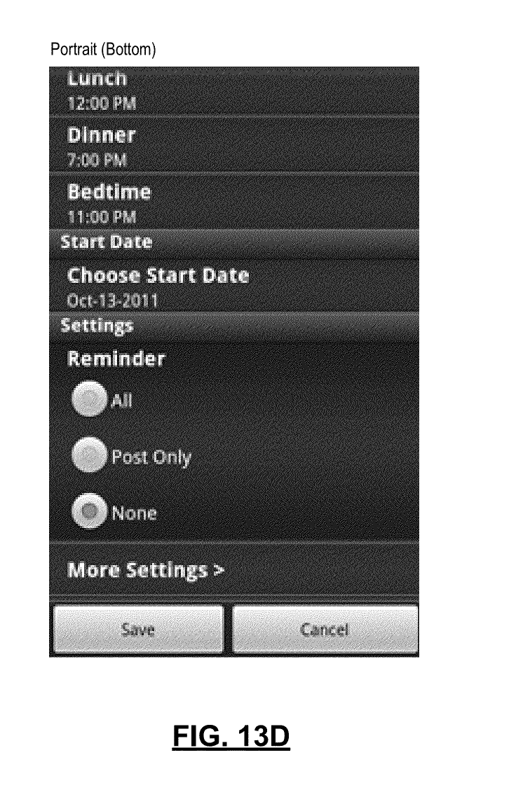

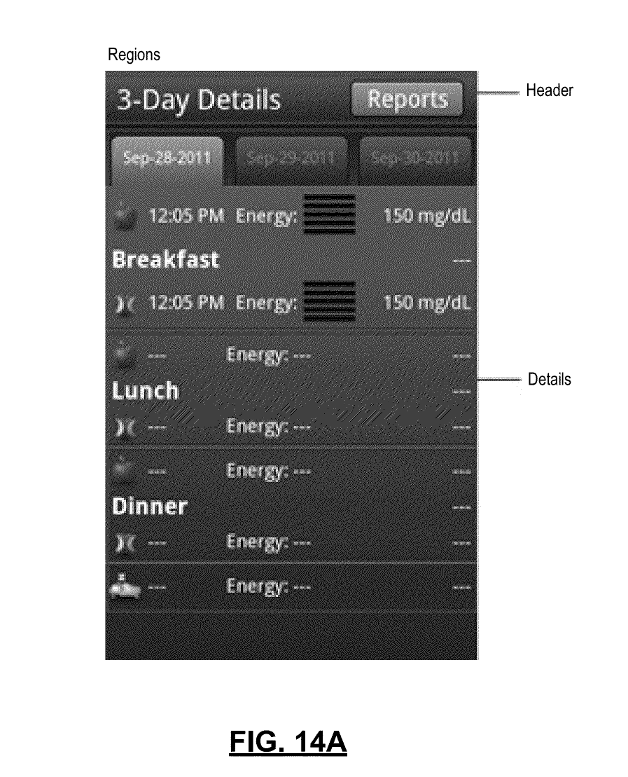

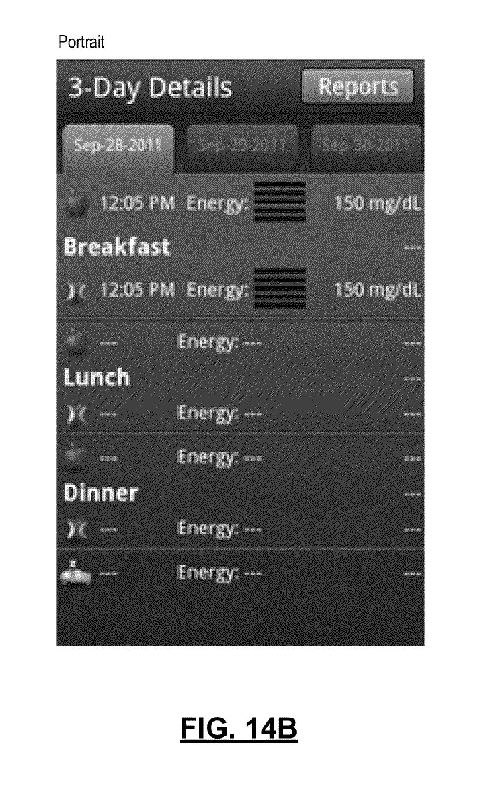

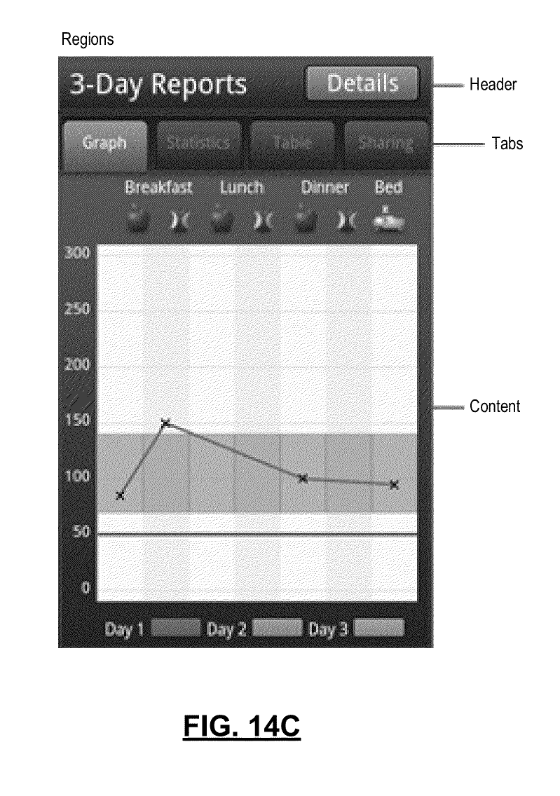

FIGS. 13A-13D represent views of a data collection module or a three-day testing protocol. The system allows queries of data related to breakfast, lunch and dinner. It also allows the user to set a reminder schedule related to chosen start date. FIGS. 14A-14C represent reports of data entered in FIGS. 11A-11F. Individual tabs can be used to distinguish various days. FIGS. 14A-14C represent detail reports which can show the status of the three-day testing protocol. Data that has been inputted is shown on the screen, while missing data is left blank. Optionally, graphs and statistics related to the input data are displayed.

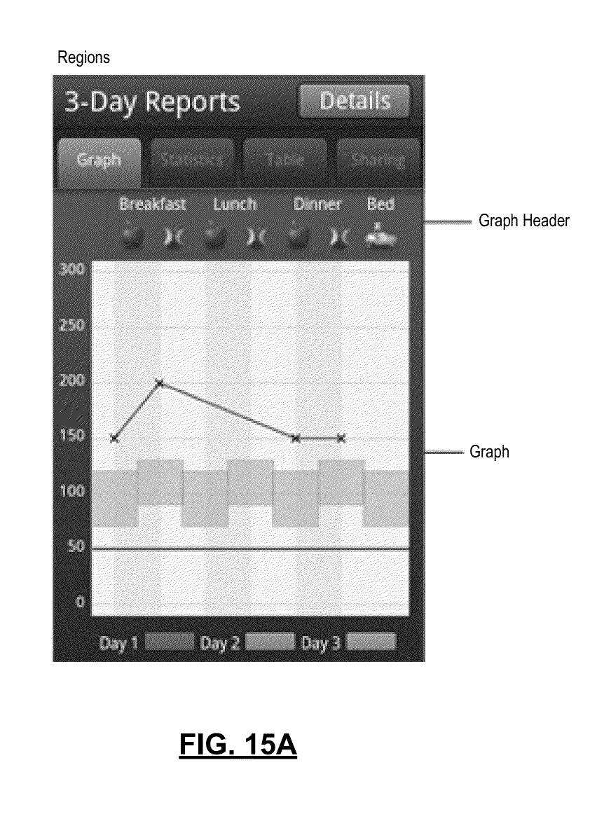

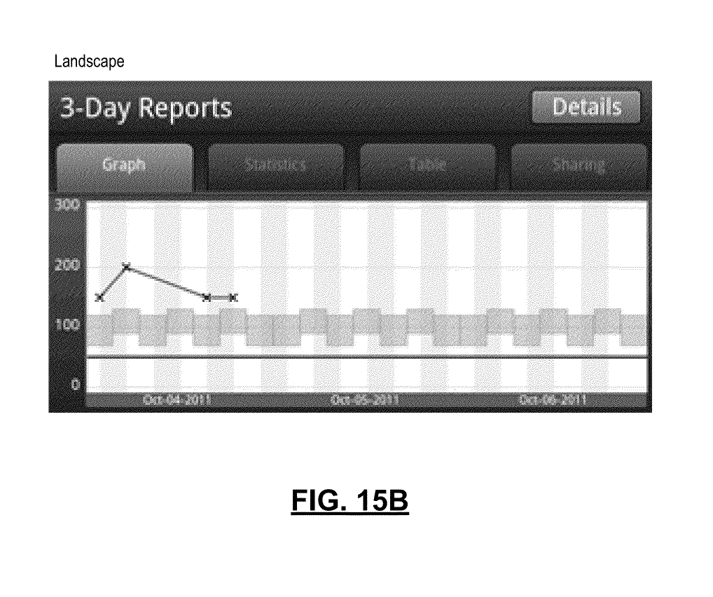

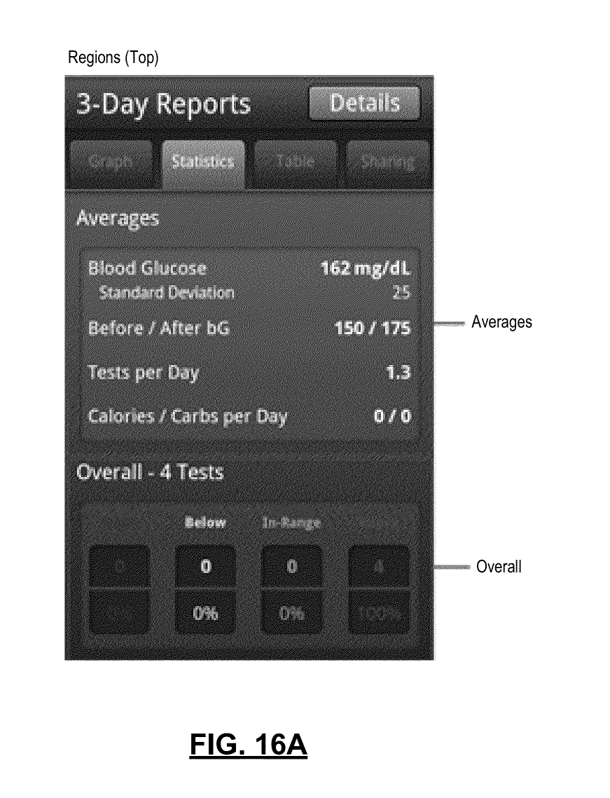

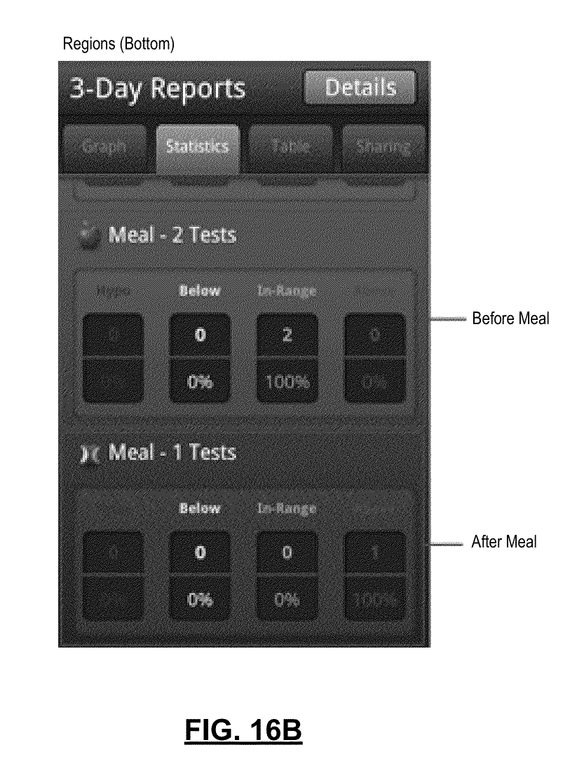

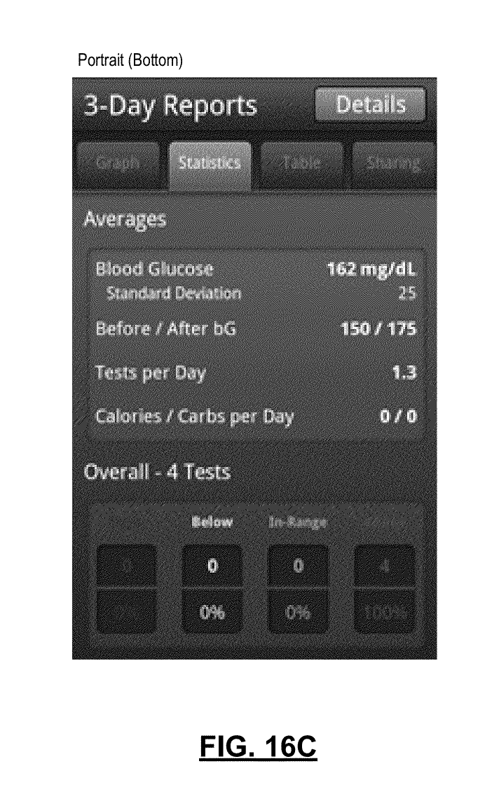

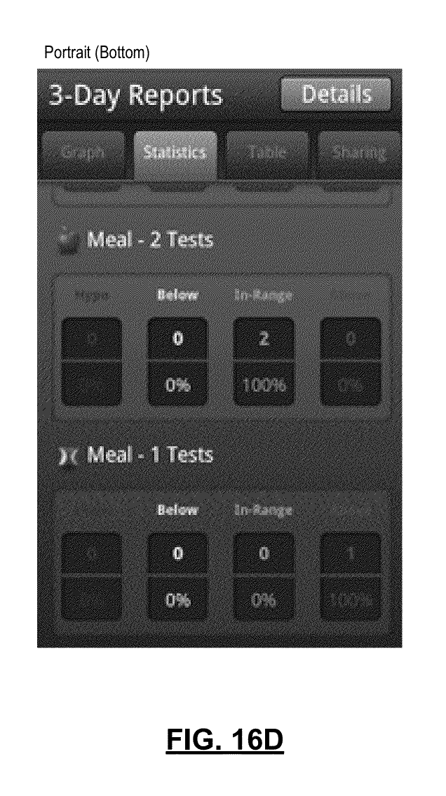

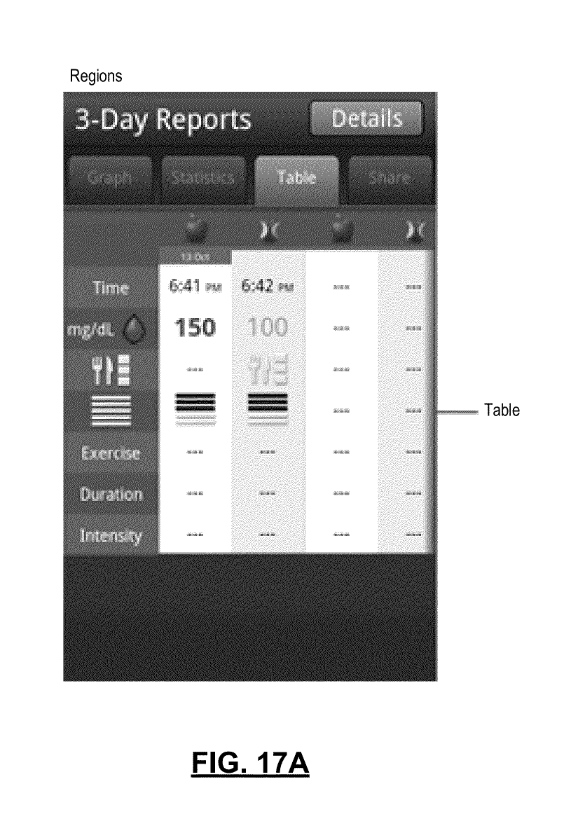

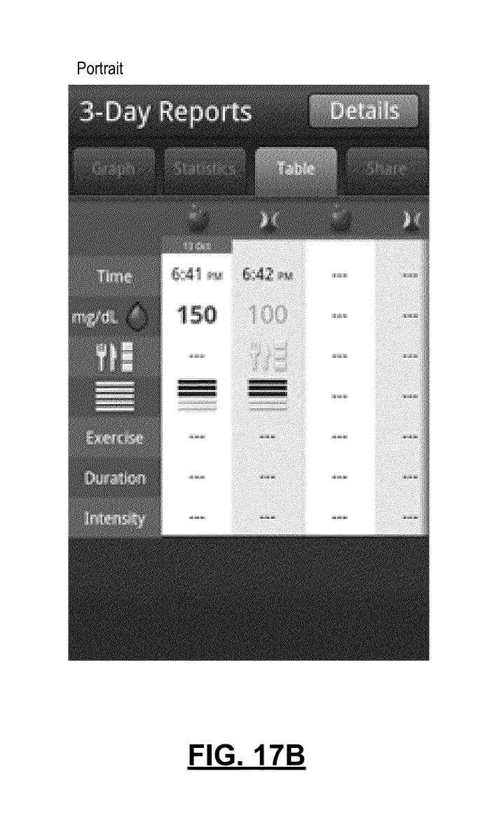

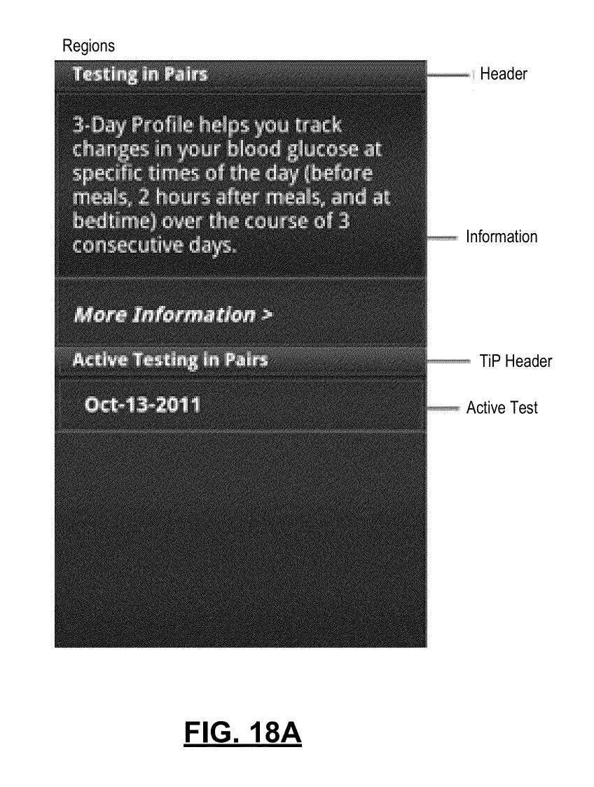

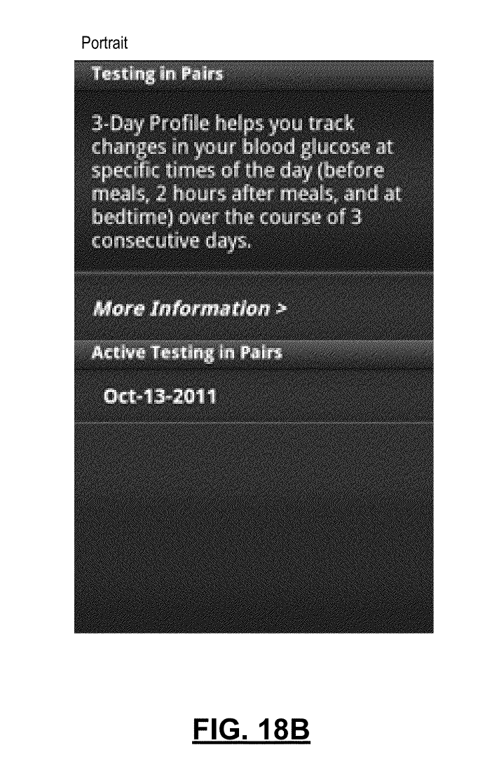

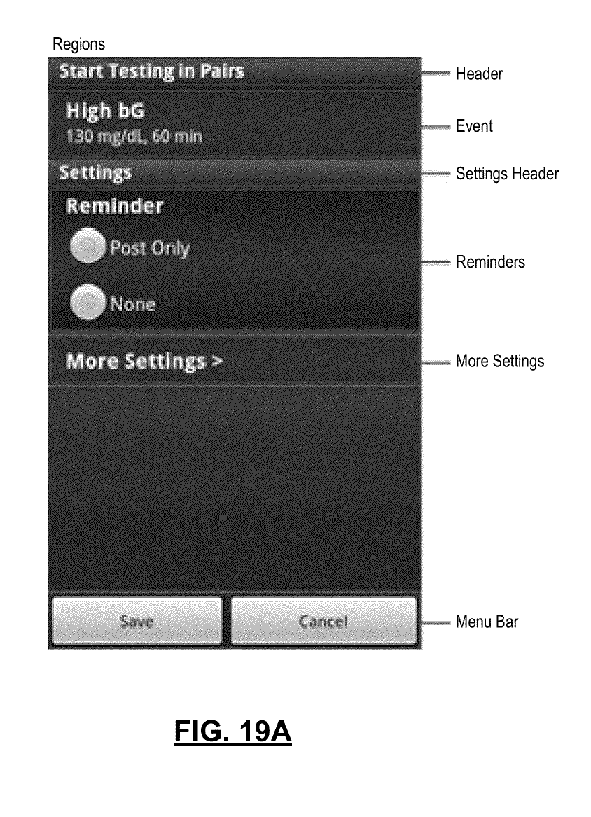

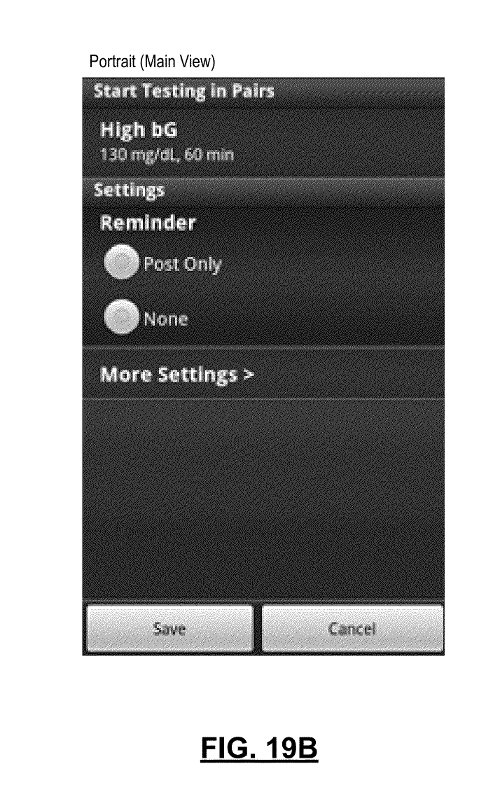

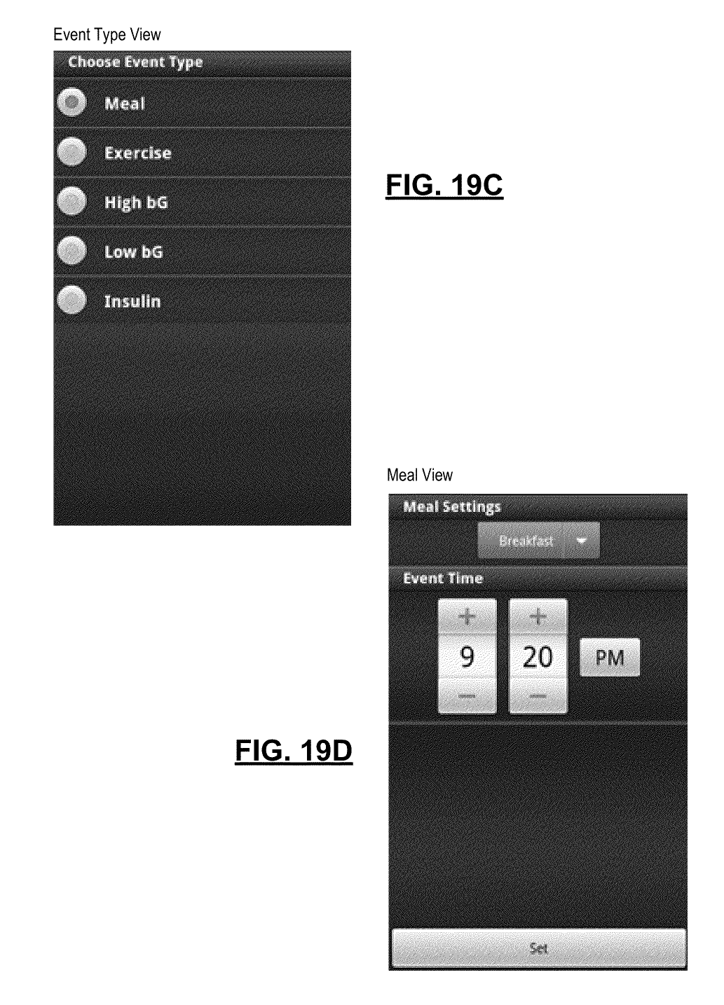

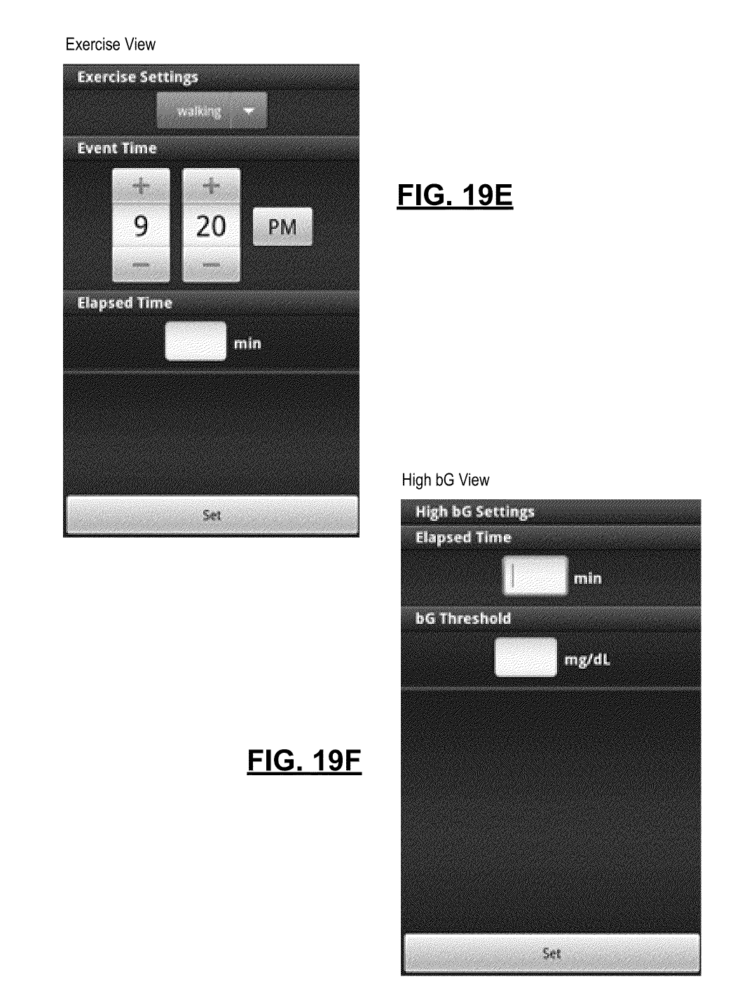

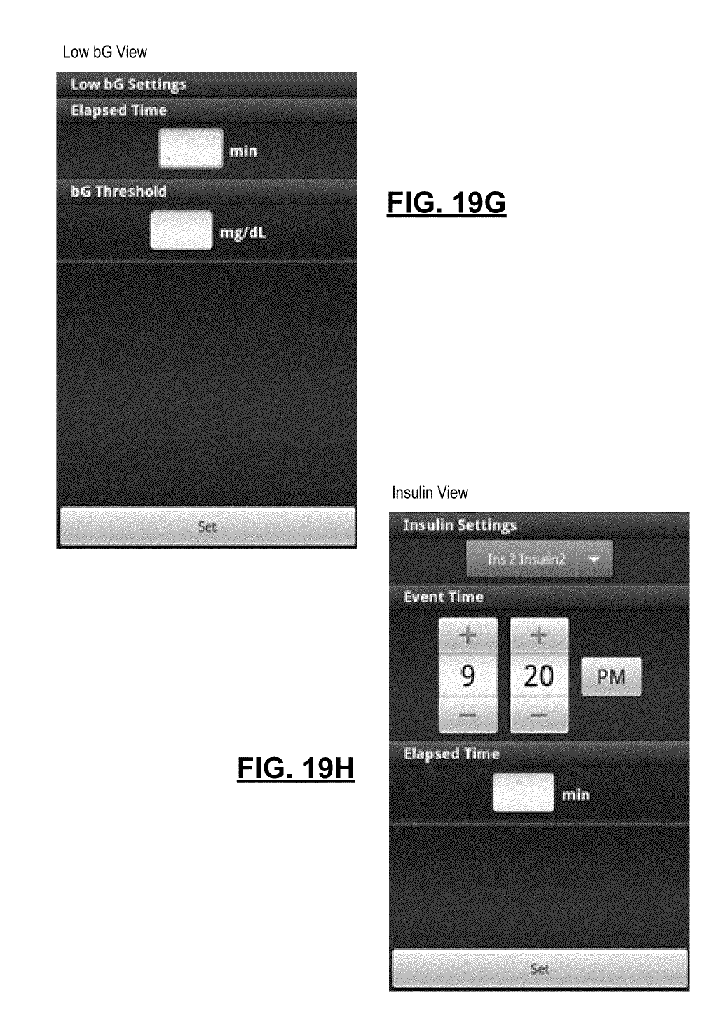

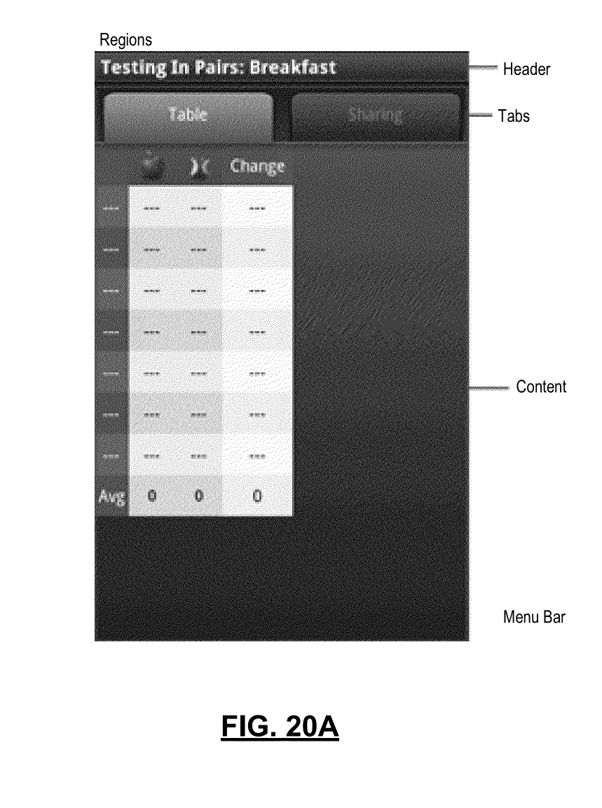

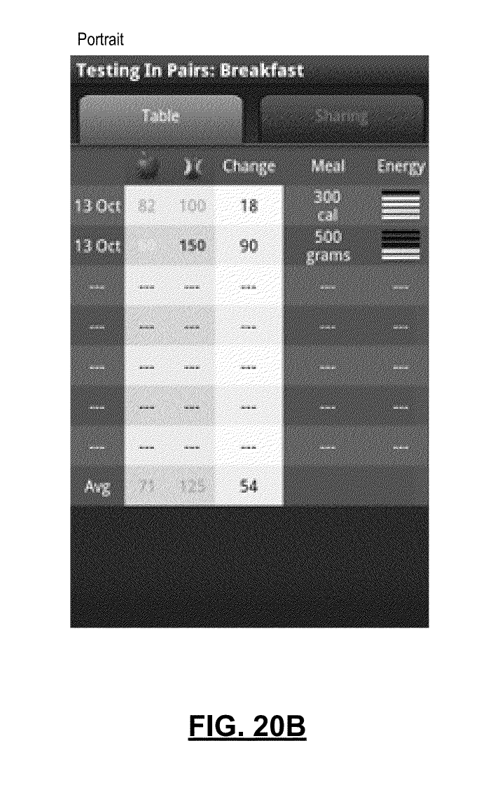

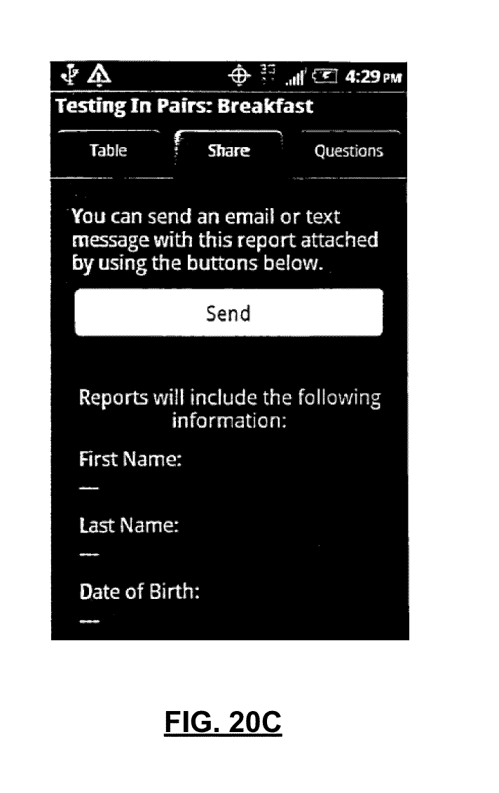

FIGS. 15A-15B represent optional graphs of bG from the report module used to inform the user of trends in their health data. The graphs can be viewed in portrait or landscape modes. The report module also can provide statistical information related to the users health conditions (see FIGS. 16A-16D). FIGS. 17A-17B represent reports shown in the form of tables. FIGS. 18A-20C represent various screens associated with the monitoring of the testing in pairs protocol. Specific event types as well as times are used to track changes in the user's condition. FIG. 20C represents the sharing of information screen for the testing in pairs module.

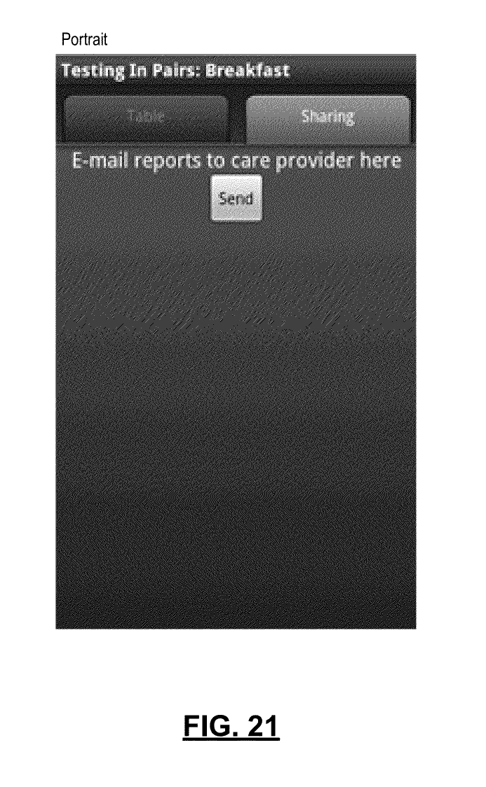

FIG. 21 represents a data sharing screen which allows the user to configure where the data from the system should be transported. This transfer, which occurs over a wireless telecom system (see FIG. 1), can be via email or the transmission to a specific IP address. Upon initiation by the user, data from the logbook is transmitted by the cellphone network to the physician or central computer.

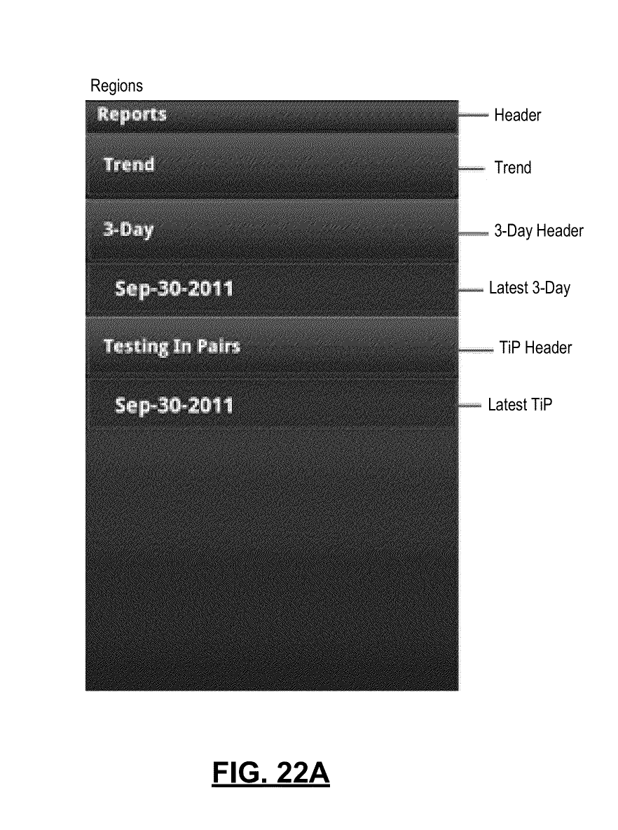

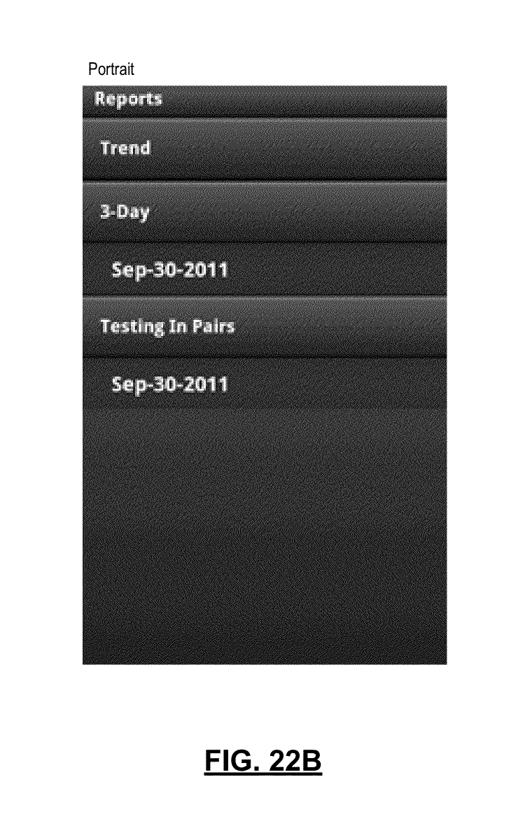

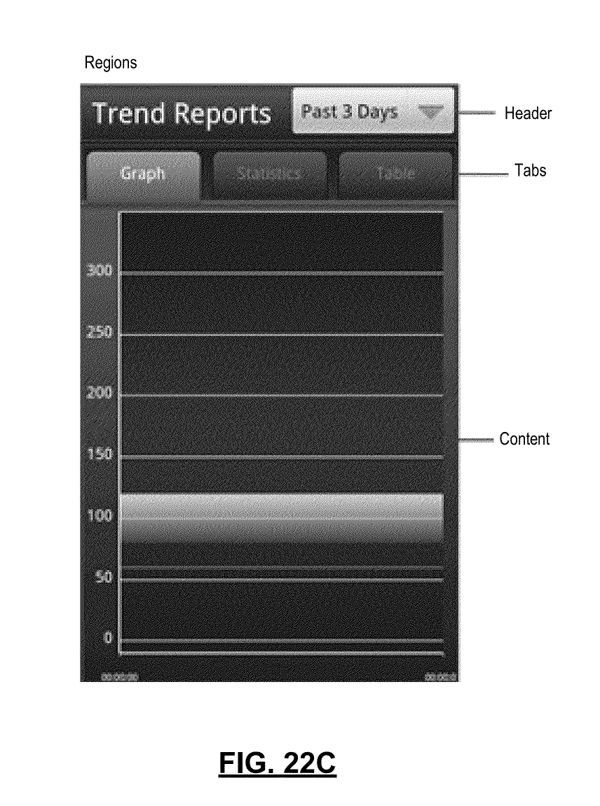

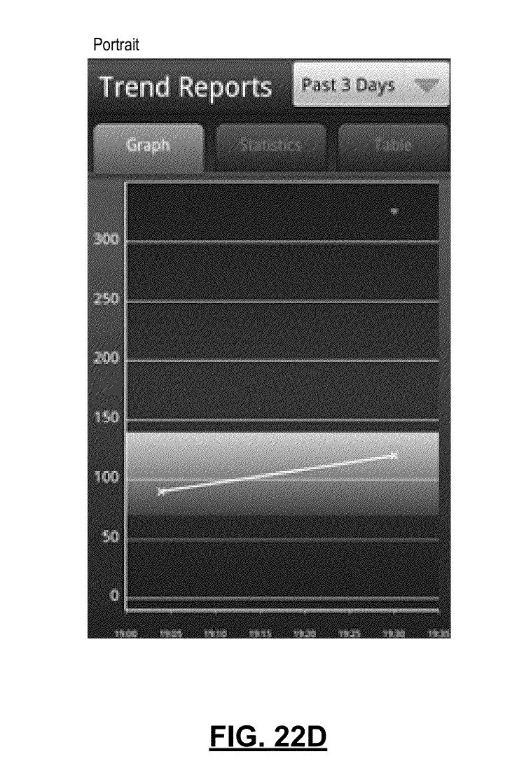

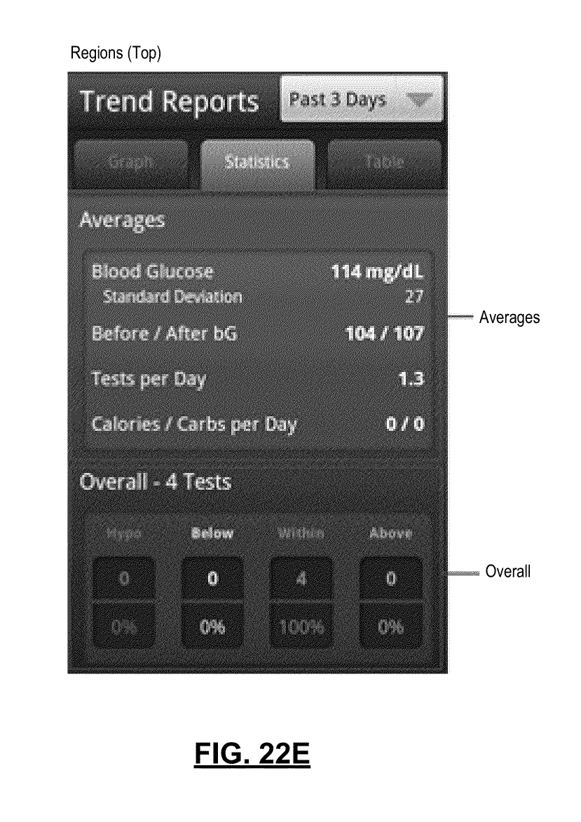

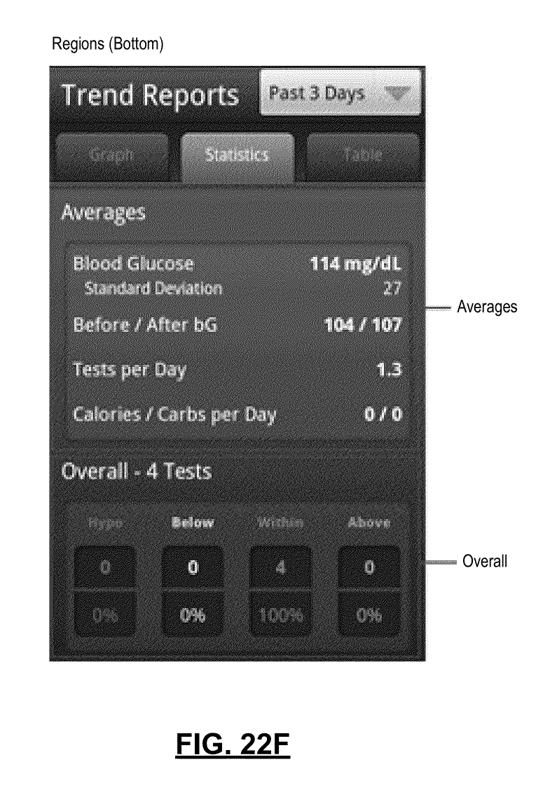

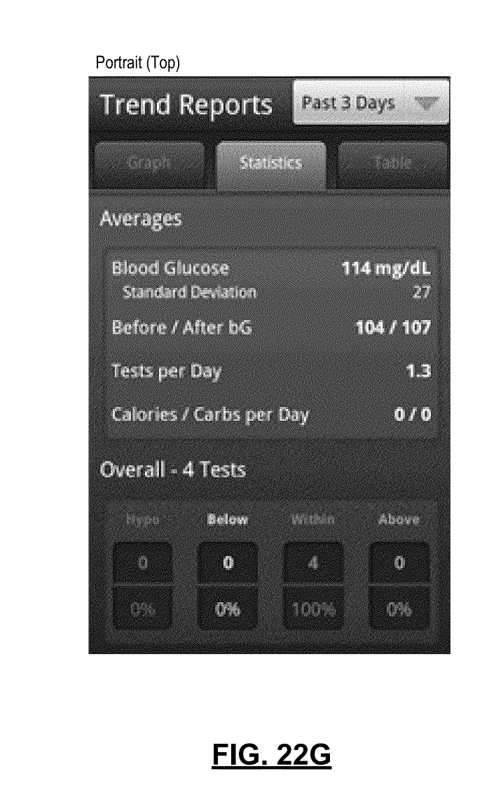

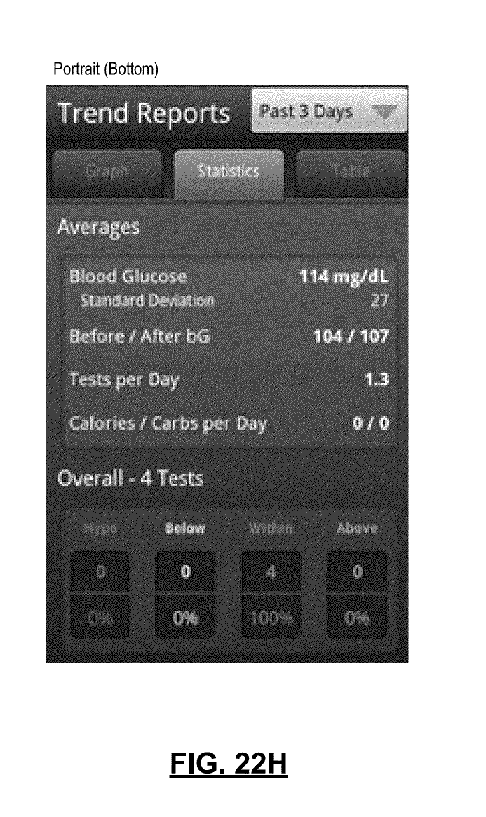

FIGS. 22A-22H represent various reporting screens for a mobile device according to the present teachings. A touch on the screen instructs the system to present the report. These reports can include trend reports in the form of graphs or tables, information which may be used by the user to define events in their lifestyle which may adversely affect their bG levels. The trend reports can include analytical statistics, such as minimum, maximum, average, or standard deviation.

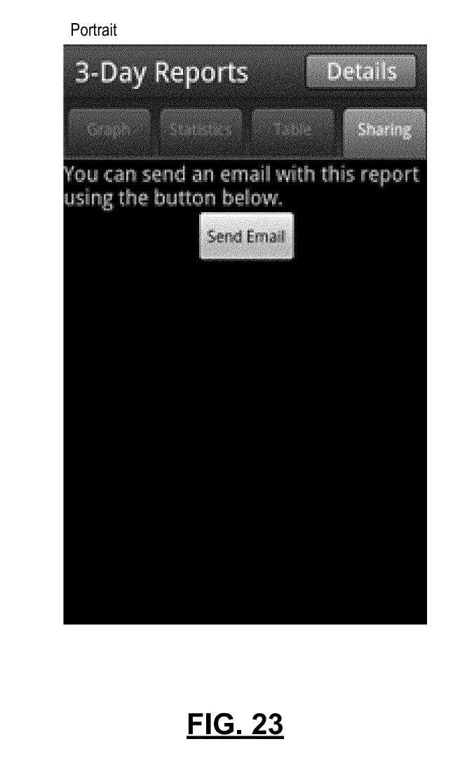

FIG. 23 represents a screen which allows the user to configure portions of the communications module for a three-day protocol. In this regard, a user is prompted to input an email address of a treating physician who is to receive medical information. This information can, for instance, include some or all of the information stored in the logbook. A touch screen button allows for sending the information.

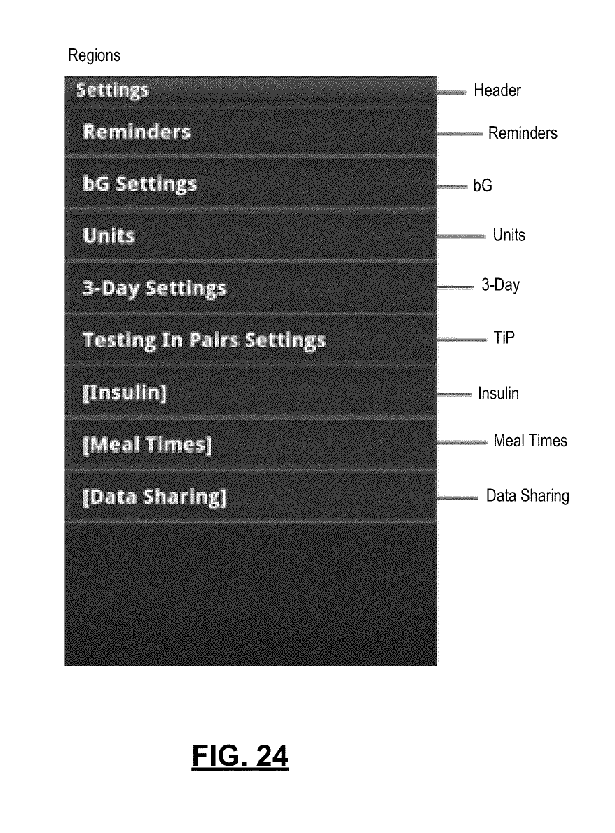

FIG. 24 represents an input page for the setting module 112. This screen allows the user to adjust the units for various user inputs. Should the units be changed by the user, the system will automatically convert data stored within the mobile device to the new units.

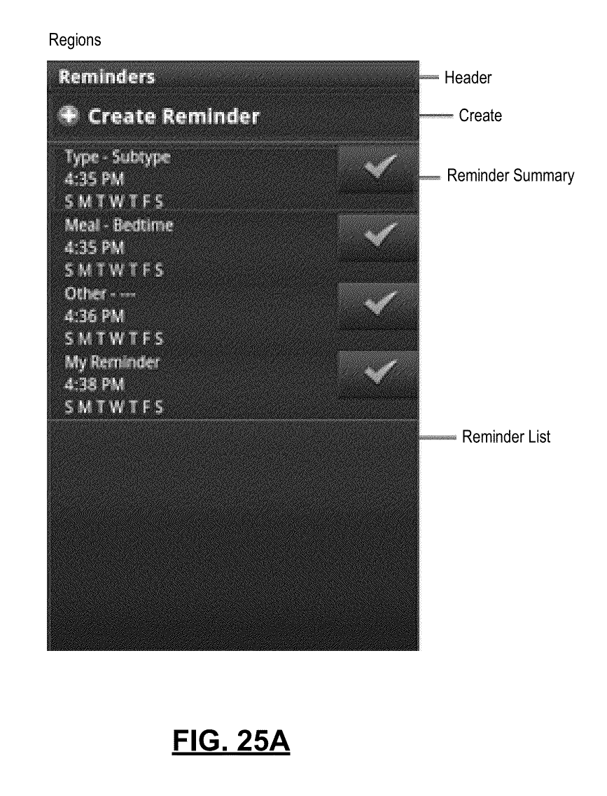

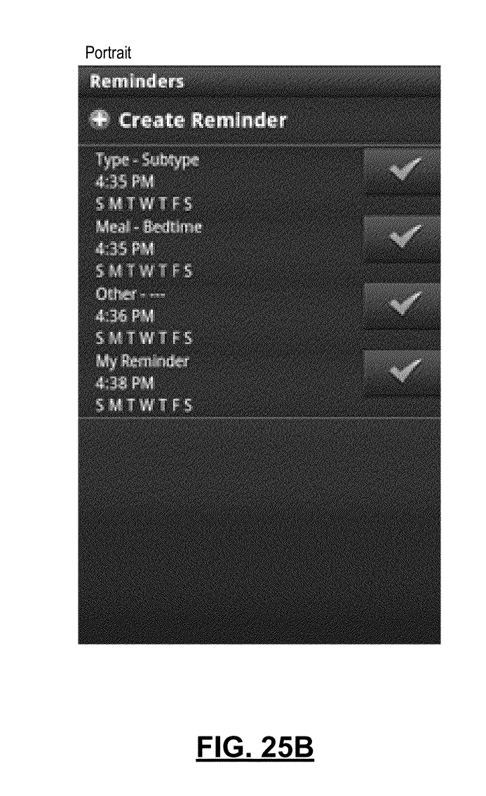

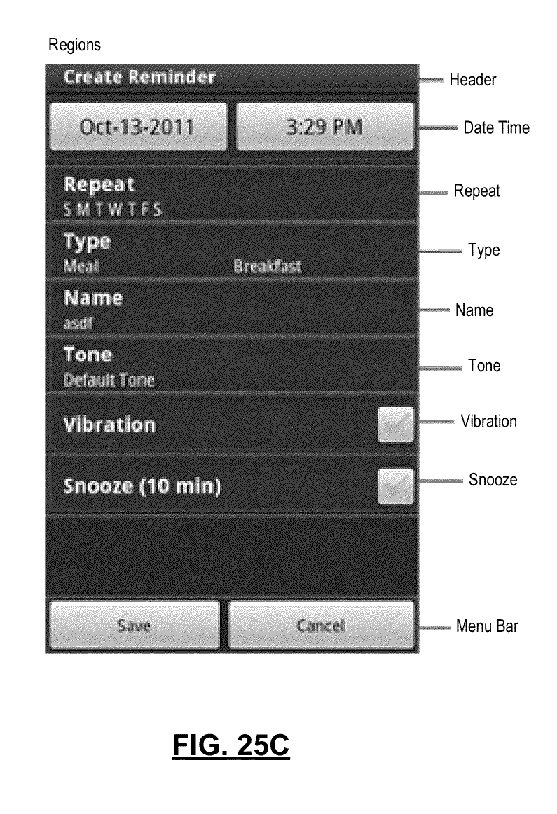

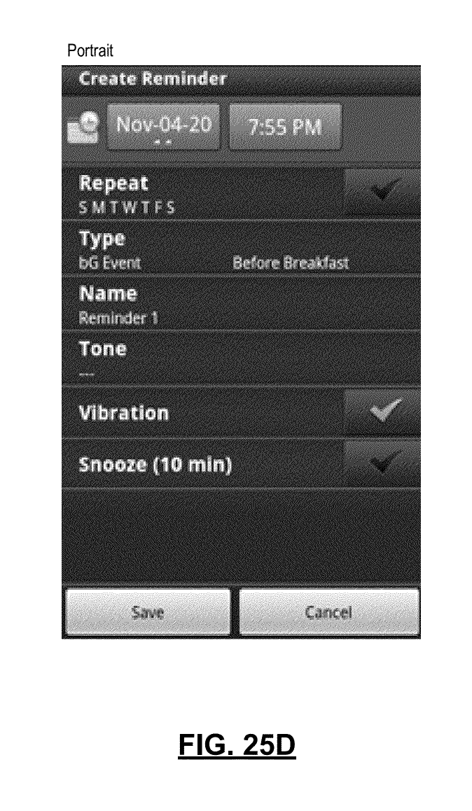

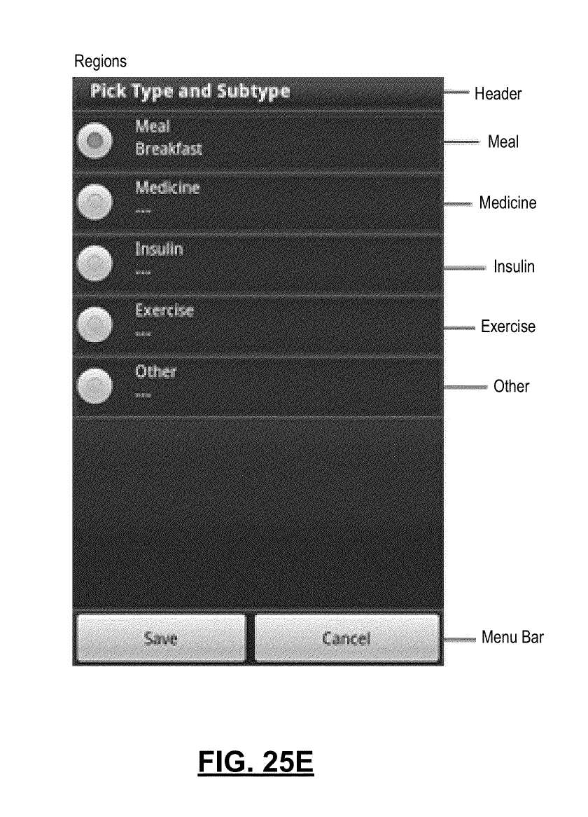

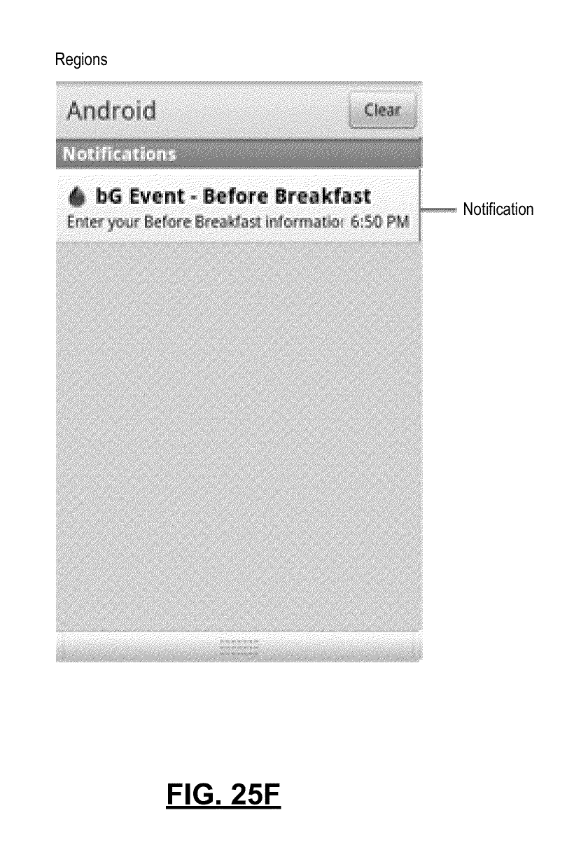

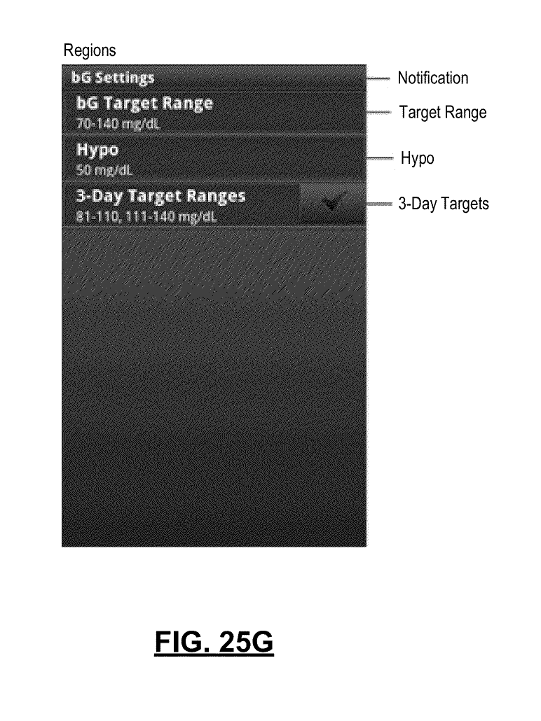

FIGS. 25A-25G represent screens related to the reminder module. These screens attempt to gain the attention of the user to inform the user that information is needed by the system. Upon acknowledgment by the user, the reminder module will prompt the user to ask if the user would like to transfer to the data entry module. In the data entry module, specific data will be collected. Reminders can be set by day or multiple times. Alarms can be set in the form of an audible or inaudible format. Additionally, snooze functions can be built in.

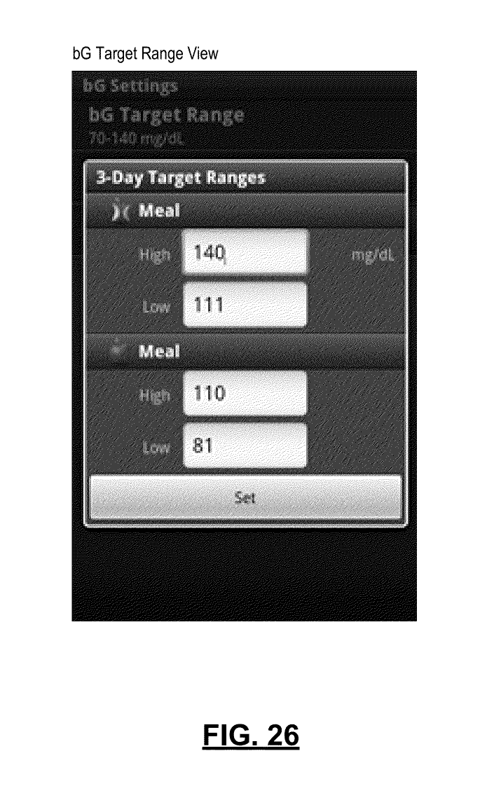

FIG. 26 represents screen for setting data sharing parameters.

As used herein, the term module may refer to, be part of, or include an Application Specific Integrated Circuit (ASIC); an electronic circuit; a combinational logic circuit; a field programmable gate array (FPGA); a processor (shared, dedicated, or group) that executes code; other suitable hardware components that provide the described functionality; or a combination of some or all of the above, such as in a system-on-chip. The term module may include memory (shared, dedicated, or group) that stores code executed by the processor.

The term code, as used above, may include software, firmware, and/or microcode, and may refer to programs, routines, functions, classes, and/or objects. The term shared, as used above, means that some or all code from multiple modules may be executed using a single (shared) processor. In addition, some or all code from multiple modules may be stored by a single (shared) memory. The term group, as used above, means that some or all code from a single module may be executed using a group of processors. In addition, some or all code from a single module may be stored using a group of memories.

The apparatuses and methods described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A mobile computing device for managing diabetes care of a patient, comprising:
a display device;
a data entry interface configured to receive glucose measures for the patient and store the glucose measures in a patient log residing on the device;
a data store that stores a plurality of structured collection procedures, where each of the structured collection procedures specifies one or more collection events for obtaining glucose measures from the patient;
a selection module that operates selectively to analyze the glucose measures in the patient log and select a given structured collection procedure from the plurality of structured collection procedures;
an administrative module in data communication with the data store and operable to prompt the patient, through the display device, to input glucose measures into the data entry interface, where the prompting of the patient is in accordance with the given structured collection procedure;
a wireless transceiver configured to transmit data through a telephone network to a treating physician associated with the patient; and
a data analysis module that operates selectively to analyze the glucose measures in the patient log and interfaces with the wireless transceiver to transmit data indicative of a patient medical condition when a level of patient medical data is outside of a predetermined window.

2. A system for managing blood glucose levels in a patient, the system comprising:
a data entry module configured to record a blood glucose level and at least one health parameter;
a display module;
a communications module having a cellphone transceiver;
a structured test module configured to run one of a three-day testing protocol, and a testing in testing pairs protocol which is stored in a memory location; and
an analysis module configured to transfer data from the data entry module to a data log module, said analysis module having a logic module configured to selectively initiate the structured testing module to run one of the testing protocols and recursively prompt a patient through the display module to input data, and in response to the input data selectively forwarding data to the communications module.

3. The system according to claim 2, wherein the display module presents a graph of data indicative of blood glucose.

4. The system according to claim 2, wherein the communications module is configured to transfer data via email, MMS, SMS, to a treating physician.

5. The system according to claim 2, wherein the logic module prompts the user for additional data based on changes in the levels of a health parameter in the data log module.

6. The system according to claim 2, wherein the at least one health parameter is selected from the group consisting of weight, physiological state, food intake, exercise, and time of nutritional intake.

7. A system for managing blood glucose levels in a patient, the system comprising:
a data entry module configured to record blood glucose level and at least one health parameter;
a setting module configured to accept user defined parameters;
a logbook module having a memory to track and memorialize changes in the blood glucose and the at least one health parameter; and
an analytics module configured to transfer data from the data entry module, the settings module, and the logbook to selectively initiate a testing protocol in response to the data.

8. The system according to claim 7, further comprising a structured testing module configured to manage a structured testing protocol.

9. The system according to claim 8, further comprising a communications module configured to transfer data from the logbook to a computer through a telecommunications network.

10. The system according to claim 7, further comprising a reminder module configured to provide reminders through a display device, the reminders requesting the user to record blood glucose and at least one health parameter through the data entry module.

11. The system according to claim 7, further comprising a display module coupled to the data entry module.

12. The system according to claim 7, wherein the testing protocol comprises a three-day testing protocol and a testing in pairs protocol.

13. The system according to claim 7, further comprising a data export module coupled to the analytics module where a user can set data sharing parameters.

14. A system for managing blood glucose levels in a patient, the system comprising:
an install module configured to install software onto a mobile device;
a setting module configured to allow a user to set a plurality of user definable parameters on the mobile device and store them in a first memory location;
a logbook configured to store medical information on the medical device in a second memory location;
a data export module configured to export health data from the second memory locations via a telecommunications system to a central computer;
a reminder module configured to prompt a user to enter health data into the mobile device;
a structured testing module configured to prompt the user to enter data into the mobile device based on a structured bG test protocol;
a reporting module configured to report data within the logbook; and
a communications module configured to transmit data from the second memory location in the logbook to a central computer.

15. The system according to claim 14, wherein the setting module is configured to accept inputs via the mobile device to set parameters for the structured tests.

16. The system according to claim 14, wherein the mobile device comprises a wireless telephone and the communications module is configured to communicate data through a wireless telephone network.

17. The system according to claim 14, wherein the reporting module produces a graph of bG over time.

18. The system according to claim 14, wherein the data log stores bG, a timing indicator, and at least one health parameter.

19. The system according to claim 14, further comprising an analysis module configured to initiate a structured test based upon a calculation which uses data stored in the second memory location in the logbook.

20. The system according to claim 14, wherein the health data comprises weight, pulse, blood pressure, and amount of exercise.
